(19) United States
(12) Patent Application Publication
(10) Pub. No.: US 2026/0183125 A1
(43) Pub. Date: Jul. 2, 2026

(54) ORTHOPAEDIC PLANNING SYSTEMS AND METHODS OF REPAIR ASSOCIATED WITH EROSION

(71) Applicant: ARTHREX, INC., Naples, FL (US)

(72) Inventors: Addison Davis Olmsted, Naples, FL (US); Kyle James Dampier, Naples, FL (US); Asheesh Bedi, Skokie, IL (US); Michael Bozeman, Naples, FL (US)

(21) Appl. No.: 19/434,149

(22) Filed: Dec. 29, 2025

Related U.S. Application Data
(60) Provisional application No. 63/740,178, filed on Dec. 30, 2024.

(51) Int. Cl. A61F 2/46; A61B 34/00; A61B 34/10; G16H 20/40

(52) U.S. Cl. CPC A61F 2/4612; A61B 34/25; G16H 20/40; A61B 2034/105; A61F 2002/4633

(57) ABSTRACT

This disclosure relates to planning systems and methods associated with erosion along an articular surface of a joint. The planning systems and methods disclosed herein may be utilized for planning orthopaedic procedures to restore functionality to a joint, may include determining an amount of erosion along or otherwise adjacent to an articular surface of a bone. One or more spherical objects may be utilized to determine the erosion.

### CROSS-REFERENCE TO RELATED APPLICATIONS

This application claims the benefit of U.S. Provisional Application No. 63/740,178, filed Dec. 30, 2024, which is incorporated herein by reference in its entirety.

### BACKGROUND

This disclosure relates to orthopaedic procedures and, more particularly, to systems and methods for determining erosion associated with an articular surface of a joint.

Many bones of the human musculoskeletal system include articular surfaces. The articular surfaces articulate relative to other bones to facilitate different types and degrees of joint movement. The articular surfaces can erode or experience bone loss over time due to repeated use or wear or may fracture as a result of a traumatic impact. These types of bone defects can cause joint instability and pain. Some techniques utilize a bone graft and/or implant to repair a defect adjacent the articular surfaces.

The bone deficiency may occur along an articular surface of a glenoid. The surgeon may treat the deficiency by positioning an implant or bone graft along the glenoid.

### SUMMARY

This disclosure relates to planning systems and methods of repair. The planning systems and methods may be utilized for planning and implementing orthopaedic procedures to restore functionality to a joint, including determining erosion adjacent an articular surface of the anatomy.

A system for planning an orthopaedic procedure according to an implementation may include a computing device including one or more processors coupled to memory. The one or more processors may be operable to collectively execute a planning environment. The planning environment may be operable to access a virtual three-dimensional glenoid model associated with a glenoid of a patient. The glenoid model may include a three-dimensional surface contour. The planning environment may be operable to fit a first three-dimensional spherical object to a first portion of the surface contour. The planning environment may be operable to fit a second three-dimensional spherical object to a second portion of the surface contour such that a volume of the first spherical object may overlap with a volume of the second spherical object. The planning environment may be operable to determine an erosion condition associated with the surface contour based on a relative size between the first and second spherical objects.

A system for planning an orthopaedic procedure according to an implementation may include a computing device including one or more processors coupled to memory. The one or more processors may be operable to collectively execute a planning environment. The planning environment may be operable to access a first virtual three-dimensional anatomical model associated with a first bone of a patient. The first virtual anatomical model may include a three-dimensional surface contour associated with a socket of a joint. The planning environment may be operable to access a second virtual three-dimensional anatomical model associated with a second bone that may cooperate with the socket of the first bone to establish the joint. The planning environment may be operable to fit a first three-dimensional spherical object to a first portion of the surface contour. The planning environment may be operable to fit a second three-dimensional spherical object to a second portion of the surface contour such that a volume of the first spherical object may overlap with a volume of the second spherical object. The planning environment may be operable to fit a third spherical object to a volume of the second anatomical model. The planning environment may be operable to determine an erosion condition associated with the surface contour based on a volume of the first and second spherical objects relative to a volume of the third spherical object.

A method of planning an orthopaedic procedure according to an implementation may include fitting a first spherical object to a first portion of a three-dimensional surface contour of a first virtual three-dimensional anatomical model. The first portion may be associated with a socket of a joint. The method may include fitting a second spherical object to a second portion of the surface contour adjacent to the first portion. The method may include determining an erosion condition associated with the surface contour based on a relative size between the first and second spherical objects. The method may include displaying, in a graphical user interface, the first and second spherical objects relative to the surface contour of the first anatomical model. The method may include displaying, in the graphical user interface, an indicator associated with the erosion condition.

The present disclosure may include any one or more of the individual features disclosed above and/or below alone or in any combination thereof.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 discloses a planning system according to an implementation.

FIG. 2 discloses a planning system including a user interface according to another implementation.

FIG. 3 discloses first and second spherical objects positioned relative to an anatomical model in the user interface of FIG. 2.

FIGS. 4-5 disclose aspects of the spherical objects relative to the anatomical model of FIG. 2.

FIG. 6 discloses a third spherical object relative to another anatomical model according to an implementation.

FIG. 7 discloses a fit between the first and second spherical objects and a surface contour of the anatomical model of FIG. 3.

FIGS. 8-9 disclose implementations of the spherical objects relative to different geometries of the anatomical model of FIG. 2.

FIGS. 10A-10B and 11 disclose intersecting ring implementations associated with the spherical objects relative to different geometries of the anatomical model of FIG. 2 and a directional indicator.

FIGS. 12-13 disclose implementations of the spherical objects relative to different amounts of erosion associated with the anatomical model of FIG. 2.

FIG. 14 discloses an implementation of determining severity of erosion based on the spherical objects relative to the anatomical model of FIG. 2.

FIG. 15 discloses a heat map associated with depth of erosion along an articular surface of the anatomical model.

FIG. 16 discloses a method of planning and executing an orthopaedic procedure.

FIGS. 17A-17B disclose perspective views of wireframes of first and second spherical objects positioned relative to a first anatomical model according to an implementation.

FIG. 18 discloses a perspective view of the wireframes of the first and second spherical objects positioned relative to the first anatomical model of FIGS. 17A-17B and a wireframe of a third spherical object positioned relative to a second anatomical model.

FIGS. 19A-19B disclose the wireframes of the first, second and third spherical objects positioned relative to the first anatomical model of FIG. 18.

FIGS. 20A-20B disclose an implant model positioned relative to an anatomical model associated with erosion.

FIG. 21 discloses an implementation of the user interface including parameters associated with erosion and a (e.g., wear) classification.

FIG. 22 discloses an image of a glenoid and a humerus including objects associated with a relative concavity.

FIG. 23 discloses another image of a glenoid and a humerus including objects associated with a relative concavity.

Like reference numbers and designations in the various drawings indicate like elements.

### DETAILED DESCRIPTION

This disclosure relates to surgical planning, systems and methods of repair. The planning systems described herein may be utilized for orthopaedic procedures and may be utilized to create, edit, execute and/or review surgical plans. The surgeon or clinical user may utilize the planning systems pre-operatively, intra-operatively and/or post-operatively. The planning systems and method disclosed herein may include determining erosion (e.g., bone loss) along an articular surface of a bone, such as bone loss along a glenoid. The planning systems may be utilized to select a surgical procedure for treating the patient, including select and/or precisely positioning an implant or bone graft, which may improve mobility and healing of the patient.

In shoulder arthroplasty, direction and severity of glenoid erosion due to humeral head misalignment may have implications for treating the patient, including procedure and/or implant selection. The classification of erosion direction has been relative to anatomic planes based on procedure. The Walch classification system is a qualitative two-dimensional (2D) classification system that may be used to classify glenoid erosion based on a single axial computerized tomography (CT) slice, giving an anterior/posterior (A/P) wear direction and severity. Other classifications such as Favard may classify erosion based on a superior/inferior (S/I) direction relative to the glenoid.

The systems and methods disclosed herein may be utilized to classify and/or quantify glenoid erosion in two and/or three dimensions. Overall glenoid deformity associated with misalignment of the humeral head may be determined. The disclosed techniques may be utilized to determine an (e.g., absolute) three-dimensional (3D) direction and/or severity of erosion on the glenoid. The surgeon or clinical user may evaluate erosion relative to two or more slices associated with imagery of the anatomy.

A set of spherical objects may be positioned relative to a surface contour of the articular surface to determine an erosion condition, including erosion location (e.g., direction) and/or severity (e.g., magnitude). The spherical objects may overlap to establish an overlapping (e.g., double) spherical object.

One or more indicators, such as humeral head location (e.g., subluxation), overall glenoid version and/or inclination, and/or the centers and/or radii of the spherical objects may be utilized to make one or more determinations relating to erosion. The disclosed techniques may be utilized to make one or more of the following determinations: (1) whether erosion is present on the glenoid surface; (2) whether the erosion may be central on the glenoid surface or may be peripheral to the glenoid surface (e.g., due to misalignment of the humeral head); (3) if the erosion is peripheral, which of the spherical objects may be representative of the paleo-glenoid surface and/or which of the spherical objects may be representative of the neo-glenoid surface; (4) if the erosion is peripheral, a location (e.g., direction) of the erosion relative to the glenoid face; and/or (5) a severity (e.g., magnitude) of the erosion. The disclosed techniques may be utilized to determine erosion along the articular surfaces of various bones and joints, including the shoulder, hip, ankle, wrist, hand or knee.

The location (e.g., direction) and/or severity (e.g., magnitude) of glenoid erosion may be determined utilizing one or more of the following steps. Surface points along the glenoid may be determined. The surface points may be identified based on one or more images of the anatomy, such as a segmented CT scan. The surface points may be utilized to establish a spline and/or 3D surface contour. A set of spherical objects may be fit to the surface points. The spherical objects may be initialized utilizing various techniques. A radius of each spherical object may be initialized to a radius of an overall best fit sphere of the glenoid surface. The centers of each spherical object may be positioned a distance away from a glenoid (e.g., best fit) plane. The distance may be equal to the selected radius. The centers of the spherical objects may be equidistant from the anterior and posterior most aspect of the glenoid. The spherical objects may overlap to establish a double intersecting spherical object, which may be fit to the 3D surface contour.

The spherical objects may be fit to the articular surface of the anatomy, such as the glenoid, to assess (e.g., overall) erosion. The double intersecting spherical object may be representative of the native glenoid surface and erosion, which may be associated with humeral head misalignment. A geometry of the native (e.g., paleo) glenoid surface may be represented by a section of a first spherical surface. A portion of the humeral head that may articulate with the glenoid may be represented by a section of a second spherical surface. Erosion due to misalignment of the humerus may be associated with an imprint of the humeral head on the glenoid surface. A geometry of the imprint may be substantially equal to a geometry of the second spherical surface associated with the humeral head. One of the spherical surfaces on the glenoid may be a native (e.g., paleo) glenoid surface, and another of the spherical surfaces on the glenoid may be a neo-glenoid surface associated with erosion by the humerus. One of the spherical objects may have a geometry that may approximate a native curvature of the articular surface prior to the erosion (e.g., bone loss). Another one of the spherical objects may have a geometry that may approximate a curvature of the articular surface associated with the bone loss. Erosion associated with misalignment of the humerus may be associated with a single direction; the glenoid surface including the erosion may be represented by a total of two spherical objects.

The disclosed techniques may be utilized to determine whether erosion is present on the articular (e.g., glenoid) surface. The determination may include evaluating parameters of the spherical objects, including the radius and/or center of the respective objects. It may be determined that directional erosion (e.g., wear) may not be present on the glenoid surface. If one of the spherical objects is extremely large or extends very far from the (e.g., shoulder) joint space, then it may be determined that the articular surface may be best approximated as a single sphere fit, or the approximation made by the double sphere fit may be associated with outlier points (e.g., osteophytes or far rim points) rather than an overall curvature of the articular surface. If there is little or no erosion, one spherical object may be very large or very small due to the outlier points rather than the surface contour (e.g., less than ⅓× or greater than 2× than a size of the humeral head). On the other hand, directional erosion may be determined in response to determining that one of the spherical objects may be of a similar size as a spherical object fit to an adjacent bone (e.g. humeral head).

Various techniques for fitting the spherical objects may be utilized. An objective function for minimization may be used for fitting the overlapping (e.g., double) spherical object where the cost may be equal to the distance between points along the articular surface and the double spherical object. In implementations, an outer periphery (e.g., border) of the spherical object may be fit to a surface contour of the articular surface. The cost of the objective function may be the sum of the lengths of the lines from the double intersecting spherical object to the respective points along the surface contour of the articular surface. A least squares minimization may be applied to the cost function to determine a (e.g., best) fit double intersecting spherical object relative to the surface contour.

From the best fit double intersecting (e.g., overlapping) spherical object associated with the glenoid, erosion detection may be based on the radius and/or diameter of the spherical objects relative to the radius and/or diameter of the humeral head spherical object. The humeral head spherical object and/or glenoid version and/or inclination may be used to identify the neo and paleo (e.g., the eroded and native) glenoid surfaces. The erosion direction may be determined based on a vector projected from the center of the spherical object associated with the native glenoid surface to the center of the spherical object associated with the neo-glenoid surface. The vector may be projected onto the glenoid plane. Erosion severity may be measured by a distance of the eroded glenoid surface to the native best fit spherical object. The surgeon or clinical user may interact with the user interface to adjust one or more parameters of the spherical objects, including the size and/or position relative to the anatomical model. The determined erosion may dynamically update in response to adjusting the parameter(s) of the spherical object(s).

The surgeon or clinical user may interact with the system (e.g., preoperatively) to determine how to treat a patient. A surgical plan may be established based on the determined erosion. The surgeon or clinical user may determine which procedure and/or implant to select (e.g., total, reverse, augment) based on the determined erosion, which may be associated with the surgical plan. In implementations, the surgical plan may include dimensions and/or placement of a bone graft on the anatomy based on the determined erosion.

A system for planning an orthopaedic procedure according to an implementation may include a computing device including one or more processors coupled to memory. The one or more processors may be operable to collectively execute a planning environment. The planning environment may be operable to access a virtual three-dimensional glenoid model associated with a glenoid of a patient. The glenoid model may include a three-dimensional surface contour. The planning environment may be operable to fit a first three-dimensional spherical object to a first portion of the surface contour. The planning environment may be operable to fit a second three-dimensional spherical object to a second portion of the surface contour such that a volume of the first spherical object may overlap with a volume of the second spherical object. The planning environment may be operable to determine an erosion condition associated with the surface contour based on a relative size between the first and second spherical objects.

In any implementations, the planning environment may be operable to instantiate the first and second spherical objects such that respective centers of the first and second spherical objects may be distributed in a first direction relative to the glenoid model.

In any implementations, the first direction may be an anterior-posterior direction.

In any implementations, the planning environment may be operable to fit the first and second spherical objects, which may occur in response to adjusting a respective radius or position to reduce a distance between adjacent surface points along the surface contour and the first and second spherical objects.

In any implementations, the planning environment may be operable to associate a most medial one of the first and second spherical objects with erosion along the surface contour.

In any implementations, the planning environment may be operable to access a virtual three-dimensional humerus model associated with a humeral head of the patient. The planning environment may be operable to determine a first distance between a center of the first spherical object and a center of the humerus model. The planning environment may be operable to determine a second distance between a center of the second spherical object and the center of the humerus model. The planning environment may be operable to associate one of the first and second spherical objects with erosion along the surface contour corresponding to a lesser of the first and second distances.

In any implementations, the planning environment may be operable to determine the erosion condition based on the relative volume between the first and second spherical objects being below a first preselected volume threshold.

In any implementations, the planning environment may be operable to access a virtual three-dimensional humerus model associated with a humeral head of the patient. The planning environment may be operable to fit a third three-dimensional spherical object to the humerus model. The planning environment may be operable to determine the erosion condition in response to a relative volume between the second and third spherical objects meeting a second preselected volume threshold.

In any implementations, the planning environment may be operable to determine a relative concavity associated with a profile of the surface contour of the glenoid model. The relative concavity may be defined as a radius of the third three-dimensional spherical object divided by a radius of one of the first and second spherical objects. The planning environment may be operable to display the relative concavity in a graphical user interface.

In any implementations, the planning environment may be operable to determine a wear classification based on the relative concavity. The planning environment may be operable to display the wear classification in the graphical user interface.

In any implementations, the planning environment may be operable to determine a glenoid plane relative to the glenoid model. The glenoid plane may be associated with a profile of the glenoid. The planning environment may be operable to determine a location of erosion along the surface contour associated with the erosion condition relative to the glenoid plane.

In any implementations, the planning environment may be operable to generate an intersecting ring along an intersection between a periphery of the first spherical object and a periphery of the second spherical object. The planning environment may be operable to determine the location of the erosion based on an orientation of the intersecting ring relative to the glenoid plane.

In any implementations, the planning environment may be operable to determine a vector from a center of the first spherical object to a center of the second spherical object. The planning environment may be operable to project the vector onto the glenoid plane. The planning environment may be operable to determine a direction of the erosion based on the projected vector.

In any implementations, the first portion of the surface contour may be associated with a native glenoid surface. The second portion of the surface contour may be associated with an eroded glenoid surface. The planning environment may be operable to determine a magnitude of the erosion based on distances between respective surface points along the second portion of the surface contour and a periphery of the first spherical object.

In any implementations, the planning environment may be operable to determine a magnitude of the erosion based on a distance between a first point on a periphery of the first spherical object and a second point on a periphery of the second spherical object.

In any implementations, the planning environment may be operable to display, in a graphical user interface, the first spherical object and the second spherical object relative to the surface contour of the glenoid model.

In any implementations, the planning environment may be operable to display, in the graphical user interface, an indicator associated with a direction or magnitude of erosion associated with the erosion condition.

In any implementations, the planning environment may be operable to determine a depth of erosion along respective regions of the glenoid model based on the first and second spherical objects. The planning environment may be operable to display, in a graphical user interface, a heat map associated with the depth of erosion.

A system for planning an orthopaedic procedure according to an implementation may include a computing device including one or more processors coupled to memory. The one or more processors may be operable to collectively execute a planning environment. The planning environment may be operable to access a first virtual three-dimensional anatomical model associated with a first bone of a patient. The first virtual anatomical model may include a three-dimensional surface contour associated with a socket of a joint. The planning environment may be operable to access a second virtual three-dimensional anatomical model associated with a second bone that may cooperate with the socket of the first bone to establish the joint. The planning environment may be operable to fit a first three-dimensional spherical object to a first portion of the surface contour. The planning environment may be operable to fit a second three-dimensional spherical object to a second portion of the surface contour such that a volume of the first spherical object may overlap with a volume of the second spherical object. The planning environment may be operable to fit a third spherical object to a volume of the second anatomical model. The planning environment may be operable to determine an erosion condition associated with the surface contour based on a volume of the first and second spherical objects relative to a volume of the third spherical object.

In any implementations, the planning environment may be operable to generate an indicator associated with the erosion condition. The planning environment may be operable to display the indicator in a graphical user interface.

In any implementations, the planning environment may be operable to display, in a graphical user interface, the first spherical object and the second spherical object relative to the surface contour of the first anatomical model.

In any implementations, the planning environment may be operable to determine a reference plane relative to the first anatomical model. The reference plane may be associated with a periphery of the socket. The planning environment may be operable to determine a location of erosion along the surface contour associated with the erosion condition relative to the reference plane.

In any implementations, the planning environment may be operable to generate an intersecting ring along an intersection between a periphery of the first spherical object and a periphery of the second spherical object. The planning environment may be operable to determine the location of the erosion based on an orientation of the intersecting ring relative to the reference plane.

In any implementations, the planning environment may be operable to determine a vector from a center of the first spherical object to a center of the second spherical object. The planning environment may be operable to project the vector onto the reference plane. The planning environment may be operable to determine a direction of the erosion based on the projected vector.

In any implementations, the first anatomical model may be associated with a glenoid of the joint. The second anatomical model may be associated with a humeral head of the joint.

A method of planning an orthopaedic procedure according to an implementation may include fitting a first spherical object to a first portion of a three-dimensional surface contour of a first virtual three-dimensional anatomical model. The first portion may be associated with a socket of a joint. The method may include fitting a second spherical object to a second portion of the surface contour adjacent to the first portion. The method may include determining an erosion condition associated with the surface contour based on a relative size between the first and second spherical objects. The method may include displaying, in a graphical user interface, the first and second spherical objects relative to the surface contour of the first anatomical model. The method may include displaying, in the graphical user interface, an indicator associated with the erosion condition.

In any implementations, the steps of fitting the first and second spherical objects may include adjusting a radius or position of the first or second spherical objects to reduce a distance between surface points along the surface contour and the first or second spherical objects.

In any implementations, the method may include determining a reference plane relative to a rim of the first anatomical model associated with a periphery of the socket. The method may include determining a location of erosion along the surface contour associated with the erosion condition relative to the reference plane.

In any implementations, the method may include generating an intersecting ring along an intersection between a periphery of the first spherical object and a periphery of the second spherical object. The method may include determining the location of the erosion based on an orientation of the intersecting ring relative to the reference plane.

In any implementations, the method may include determining a vector from a center of the first spherical object to a center of the second spherical object. The method may include projecting the vector onto the reference plane. The method may include determining a direction of the erosion based on the projected vector.

In any implementations, the method may include determining a magnitude of the erosion based on distances between respective surface points along the second portion of the surface contour and a periphery of the first spherical object.

In any implementations, the indicator may be associated with a direction or a magnitude of erosion associated with the erosion condition.

In any implementations, the method may include displaying, in the graphical user interface, a heat map associated with a depth of erosion along respective regions of the surface contour based on the determined erosion condition.

In any implementations, the method may include selecting an implant for treating the joint based on the determined erosion condition.

In any implementations, the method may include positioning a three-dimensional virtual implant model associated with the selected implant relative to the surface contour based on the determined erosion condition.

In any implementations, the method may include determining which of the first and second spherical objects may be most medial relative to the first anatomical model. The method may include associating a most medial one of the first and second spherical objects with erosion along the surface contour.

In any implementations, the method may include fitting a third spherical object to a three-dimensional surface contour of a second virtual three-dimensional anatomical model. The second anatomical model may be associated with a bone of the joint. The step of determining the erosion condition may include determining whether a difference between a volume of the third spherical object and a volume of the first spherical object is within a first preselected limit. The step of determining the erosion condition may include determining whether a difference between the volume of the third spherical object and a volume of the second spherical object is within a second preselected limit.

In any implementations, the method may include determining whether the first spherical object or the second spherical object may be associated with erosion along the surface contour based on distance between respective centers of the first and second spherical objects and a center of the third spherical object.

In any implementations, the first anatomical model may be associated with a glenoid of the joint. The second anatomical model may be associated with a humeral head of the joint.

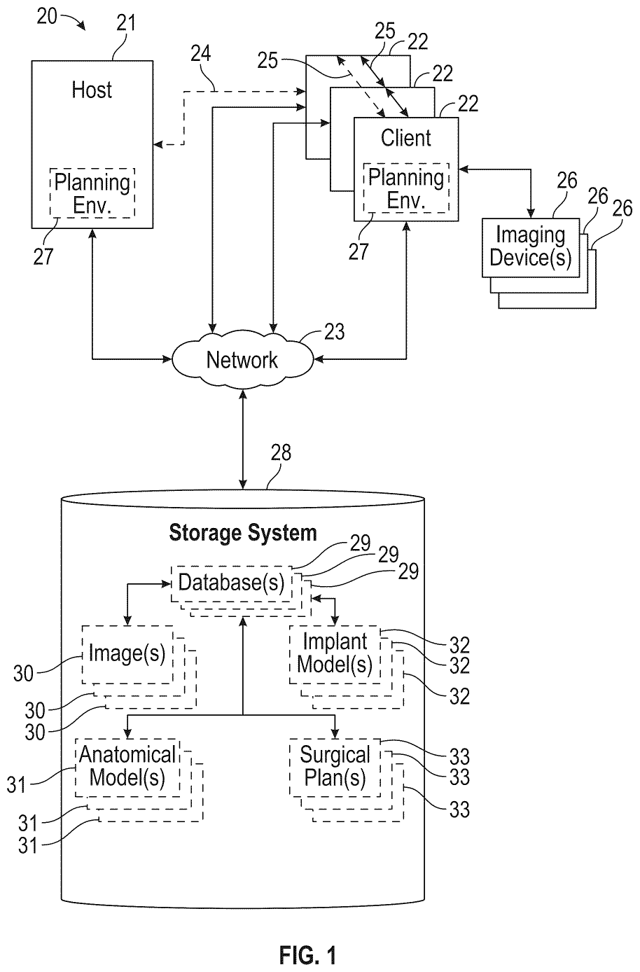

FIG. 1 discloses a surgical planning system 20 according to an implementation. The system 20 may be utilized for planning orthopaedic and/or other surgical procedures, including pre-operatively, intra-operatively and/or post-operatively to create, edit, execute and/or review surgical plans. The system 20 may be utilized for various orthopaedic and other surgical procedures, such as an arthroplasty to repair a joint. The system 20 may be utilized in the design (e.g., dimensioning) and/or placement of implant(s) and/or bone grafts, such as an implant incorporated into a shoulder prosthesis. Although the planning systems and methods disclosed herein primarily refer to repair of a glenoid during shoulder reconstruction, it should be understood that the planning system 20 may be utilized in the repair of other locations of the anatomy and other surgical procedures including repair of other bones and joints such as the hip, ankle, wrist, hand or knee.

The system 20 may include a host computer 21 and one or more client computers 22. The host computer 21 may be configured to execute one or more software programs. In implementations, the host computer 21 may be more than one computer jointly configured to process software instructions serially or in parallel.

The host computer 21 may communicate with one or more networks such as a network 23 comprised of one or more computing devices. The network 23 may be a private local area network (LAN), a private wide area network (WAN), the Internet, or a mesh network.

The host computer 21 and each client computer 22 may include one or more computer processors, memory, storage means, network devices, and input and/or output devices and/or interfaces. The input devices may include keyboards, mice and touch screens. The output devices may include monitors, speakers and printers. The memory may include UVPROM, EEPROM, FLASH, RAM, ROM, DVD, CD, a hard drive, or other computer readable medium which may store data and/or other information relating to the planning and implementation techniques disclosed herein. The host computer 21 and each client computer 22 may be a desktop computer, laptop computer, smart phone, tablet, or any other computing device. The interface may facilitate communication with the other systems and/or components of the network 23.

Each client computer 22 may be configured to communicate with the host computer 21 directly via a direct client interface 24 or over the network 23. In another implementation, the client computers 22 may be configured to communicate with each other directly via a peer-to-peer interface 25.

The system 20 may include, or may be coupled to, one or more imaging devices 26. Each client computer 22 may be coupled to one or more imaging devices 26. Each imaging device 26 may be configured to capture or acquire one or more images 30 of patient anatomy residing within a scan field (e.g., window) of the imaging device 26. The imaging device 26 may be configured to capture or acquire 2D and/or 3D greyscale and/or color images 30. Various imaging devices 26 may be utilized, such as an X-ray machine, CT machine or magnetic resonance imaging (MRI) machine that may obtain one or more images of a patient.

The client computers 22 may be configured to execute one or more software programs, including various surgical tools. Each client computer 22 may be operable to access and locally and/or remotely execute a planning environment 27. The planning environment 27 may be a standalone software package or may be incorporated into another surgical tool. The planning environment 27 may be configured to communicate with the host computer 21 either over the network 23 or directly through the direct client interface 24.

The planning environment 27 may be configured to interact with one or more of the imaging devices 26 to capture or acquire images 30 of patient anatomy. The planning environment 27 may provide a display or visualization of one or more images 30, virtual (e.g., 2D and/or 3D) anatomical (e.g., bone) models 31 and/or virtual (e.g., 2D and/or 3D) implant (or graft) models 32 via one or more graphical user interfaces (GUI). The anatomical model 31 may be representative of one or more bones and/or soft tissue. Each image 30, anatomical model 31, implant model 32 and other data and information may be stored in one or more files or records according to a specified data structure. The implant model 32 may include one or more components. The implant model 32 may be associated with various implants, such as a base (e.g., base plate) configured to be coupled to a respective articulation member (e.g., glenosphere). The articulation member and/or another portion of the implant may have an articular surface dimensioned to mate with an articular surface of an opposed bone or implant.

The system 20 may include at least one storage system 28, which may be operable to store or otherwise provide data to other computing devices. The storage system 28 may be a storage area network device (SAN) configured to communicate with the host computer 21 and/or the client computers 22 over the network 23. In implementations, the storage system 28 may be incorporated within, or may be directly coupled to, the host computer 21 and/or client computers 22. The storage system 28 may be configured to store various information, such as one or more of computer software instructions, data, database files and configurations.

In implementations, the system 20 may be a client-server architecture configured to execute computer software on the host computer 21, which may be accessible by the client computers 22 using either a thin client application or a web browser executing on the client computers 22. The host computer 21 may be operable to load the computer software instructions from local storage, or from the storage system 28, into memory and may execute the computer software using the one or more computer processors.

The system 20 may include one or more databases 29. The databases 29 may be stored at a central location, such as the storage system 28. In other implementations, one or more databases 29 may be stored at the host computer 21 and/or may be a distributed database provided by one or more of the client computers 22. Each database 29 may be a relational database configured to associate one or more images 30, anatomical models 31 and/or implant models 32 to each other and/or respective surgical plan(s) 33. Each surgical plan 33 may be associated with the anatomy of a respective patient. Each image 30, anatomical model 31, implant model 32 and/or surgical plan 33 may be assigned a unique identifier or database entry. The database 29 may be configured to store data and other information corresponding to the images 30, anatomical models 31, implant models 32 and/or surgical plans 33 in one or more database records or entries, and/or may be configured to link or otherwise associate one or more files corresponding to each respective image 30, anatomical model 31, implant model 32 and/or surgical plan 33. Images 30, anatomical models 31, implant models 32 and/or associated surgical plans 33 stored in the database(s) 29 may correspond to respective patient anatomies from prior, planned and/or hypothetical surgical cases, and may be arranged into one or more predefined categories such as sex, age, ethnicity, defect category, procedure type, surgeon, and/or facility or organization.

Each image 30 and/or anatomical model 31 may include data and other information obtained from one or more medical devices or tools, such as the imaging devices 26. The anatomical model 31 may include coordinate information relating to an anatomy of the patient obtained or derived from image(s) 30 captured or otherwise obtained by the imaging device(s) 26. Each implant model 32 may include geometry and/or coordinate information associated with a predefined design or a design established or modified by the planning environment 27. The planning environment 27 may incorporate and/or interface with one or more modeling packages, such as a computer aided design (CAD) package, to render the models 31, 32 as 2D and/or 3D volumes or constructs, which may overlay one or more of the images 30 in a display screen of a GUI.

The implant models 32 may correspond to (e.g., physical) implants and components of various configurations, shapes, sizes, procedures and/or instrumentation. The implant model 32 may be associated with a patient-specific implant for treating a single patient or may be non-patient specific (e.g., generic) for treating different patients. Each implant may include, or may otherwise be associated with, one or more components that may be situated at a surgical site including grafts and various fixation devices such as screws, anchors, nails and suture. Each implant model 32 may correspond to a single component or may include two or more components that may be configured to establish an assembly. The implant model 32 may include a base (e.g., base plate) coupled to an articulation member (e.g., glenosphere). The articulation member may have an articular surface dimensioned to mate with an articular surface of an opposed bone or implant. Each implant and associated component(s) may be formed of various materials, including metallic and/or non-metallic materials. Each virtual anatomical model 31 and/or implant model 32 may correspond to 2D and/or 3D geometry and may be utilized to generate a wireframe, mesh and/or solid construct in a display.

Each surgical plan 33 may be associated with one or more of the images 30, anatomical models 31 and/or implant models 32. The surgical plan 33 may include various parameters associated with the images 30, anatomical models 31 and/or implant models 32. The surgical plan 33 may include parameters relating to bone density and bone quality associated with patient anatomy captured in the image(s) 30. The surgical plan 33 may include parameters including spatial information relating to relative positioning and coordinate information of the selected anatomical model(s) 31 and/or implant model(s) 32.

The surgical plan 33 may include one or more revisions to an anatomical model 31 and information relating to a position of an implant model 32 relative to the original and/or revised anatomical model 31. Revisions may include removal of tissue from the anatomy, which may be performed by a cutting (e.g., drilling, sawing or reaming) operation. The surgical plan 33 may include coordinate information relating to the revised anatomical model 31 and a relative position of the implant model 32 in predefined data structure(s). Revisions to each anatomical model 31, implant model 32 and/or surgical plan 33 may be stored in the database 29 automatically and/or in response to user interaction with the system 20.

One or more surgeons and other clinical users may be provided with a planning environment 27 via the client computers 22 and may simultaneously access each image 30, anatomical model 31, implant model 32 and/or surgical plan 33 stored in the database(s) 29. Each user may interact with the planning environment 27 to create, view and/or modify various aspects of the surgical plan 33. Each client computer 22 may be configured to store local instances of the images 30, anatomical models 31, implant models 32 and/or surgical plans 33, which may be synchronized in real-time or periodically with the database(s) 29. The planning environment 27 may be a standalone software package executed on a client computer 22 or may be provided as one or more services executed on the host computer 21.

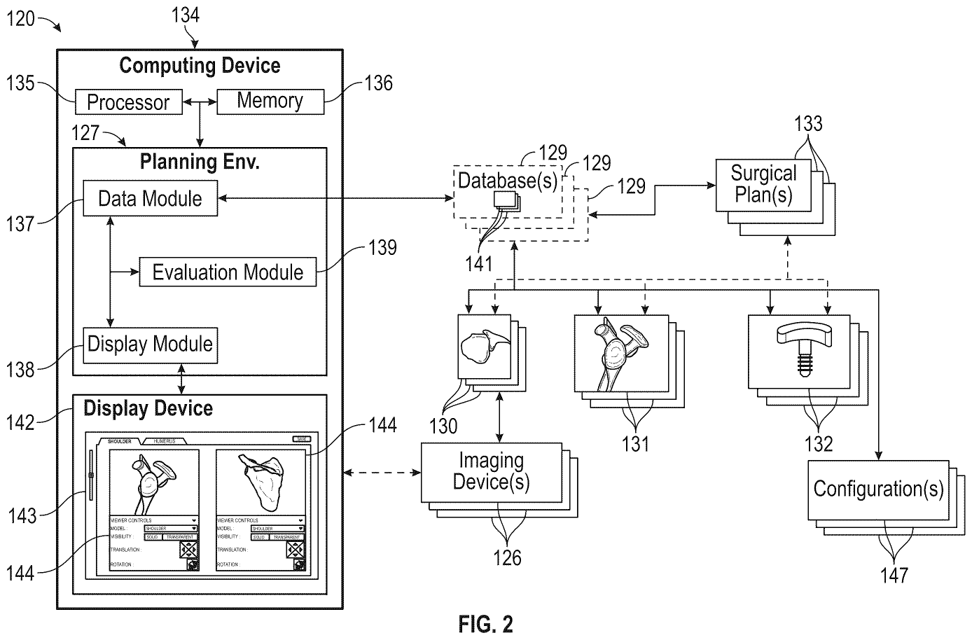

FIG. 2 discloses a planning system 120 according to another implementation. In this disclosure, like reference numerals designate like elements where appropriate and reference numerals with the addition of one-hundred or multiples thereof designate modified elements that are understood to incorporate the same features and benefits of the corresponding original elements. The system 120 may be incorporate any of the features of system 20 and/or vice versa. The system 120 may be utilized to plan and implement various orthopaedic and other surgical procedures, such as an arthroplasty to repair various bones and/or joints. The system 120 may be utilized in planning the preparation of one or more bones. The system 120 may be utilized in planning placement of implant(s) and/or bone graft(s) to restore functionality to the joint. Although the planning systems and methods disclosed herein primarily refer to repair of a glenoid associated with a shoulder joint, it should be understood that the planning system 120 may be utilized in the repair of other anatomy of the patient and other surgical procedures including repair of other joints such as a hip, ankle, wrist, hand or knee.

The system 120 may include a computing device 134 including one or more processors 135 coupled to memory 136. The computing device 134 may include any of the computing devices disclosed herein, including the host computer 21 and/or client computer 22. The processor(s) 135 may be configured to collectively execute a planning environment 127 for creating, editing, executing and/or reviewing one or more surgical plans 133 and any associated anatomical (e.g., bone) models 131 and/or implant (or graft) models 132 during pre-operative, intra-operative and/or post-operative phases of a surgery.

The planning environment 127 may include a data (e.g., interface) module 137, a display module 138 and an evaluation (e.g., spatial or comparison) module 139. Although three modules are disclosed, it should be understood that fewer or more than three modules may be utilized and/or one or more of the modules may be combined to provide the disclosed functionality.

The data module 137 may be configured to access, retrieve and/or store data and other information in the database(s) 129 corresponding to one or more images 130 of patient anatomy, anatomical model(s) 131, implant model(s) 132 and/or surgical plan(s) 133. The data and other information may be stored in one or more databases 129 as one or more records or entries 141. In implementations, the data and other information may be stored in one or more files that may be accessible by referencing one or more objects or memory locations referenced by the records 141.

The memory 136 may be configured to access, load, edit and/or store instances of one or more images 130, anatomical models 131, implant models 132, and/or surgical plans 133 in response to one or more commands from the data module 137. The data module 137 may be configured to access a virtual (e.g., 2D or 3D) anatomical model 131 from memory, such as the memory 136 and/or storage system 128. The anatomical model 131 may be associated with bone(s) and/or joint(s) of a patient. The data module 137 may be configured to cause the memory 136 to store a local instance of the image(s) 130, anatomical model(s) 131, implant model(s) 132 and/or surgical plan(s) 133, which may be synchronized with the records 141 in the database(s) 129.

The data module 137 may be configured to receive data and other information corresponding one or more images 130 of patient anatomy from various sources such as the imaging device(s) 126. The data module 137 may be configured to command the imaging device 126 to capture or otherwise acquire the image(s) 130 automatically and/or in response to user interaction.

The display module 138 may be configured to display data and other information relating to one or more surgical plans 133 in at least one graphical user interface (GUI) 143, including one or more of the images 130, anatomical models 131 and/or implant models 132. The computing device 134 may incorporate, or may be coupled to, a display device 142. The user interface 143 may include one or more display windows 144. The display module 138 may be configured to cause the display device 142 to display information in the display window(s) 144 and/or another portion of the user interface 143, including any of the information disclosed herein. A surgeon or clinical user may interact with the user interface 143 via the planning environment 127 to view one or more images 130 of patient anatomy and/or any associated anatomical models 131 and/or implant models 132. The surgeon or other user may interact with the user interface 143 via the planning environment 127 to create, edit, execute and/or review one or more surgical plans 133.

The planning system 120 may be configured to access, generate, review, edit and/or approve one or more configurations 147 associated with respective physical implant(s) and/or surgical instrument(s) (e.g., guides). The implants may be patient-specific and/or generic. The patient-specific implant may include a contour dimensioned to follow a surface contour of a bone of the patient. In implementations, the implant may include an augment portion dimensioned to fill a void (e.g., defect) in the bone, which may be associated with erosion. The implant model 132 may be representative of one or more physical implants, which may be associated with a prosthesis. The surgical instruments may be adapted for positioning one or more surgical devices (e.g., guide wires, cutting tools, etc.). Each configuration 147 may include one or more files in a predetermined data structure or format. In implementations, the configuration 147 may include a coordinate set and/or other information such as material selection(s) associated with volume(s) of the physical implant and/or surgical instrument. The physical implant(s) and/or instrument(s) may be formed utilizing various techniques, such as rapid prototyping (e.g., printing) and other additive manufacturing techniques, molding, casting and/or machining.

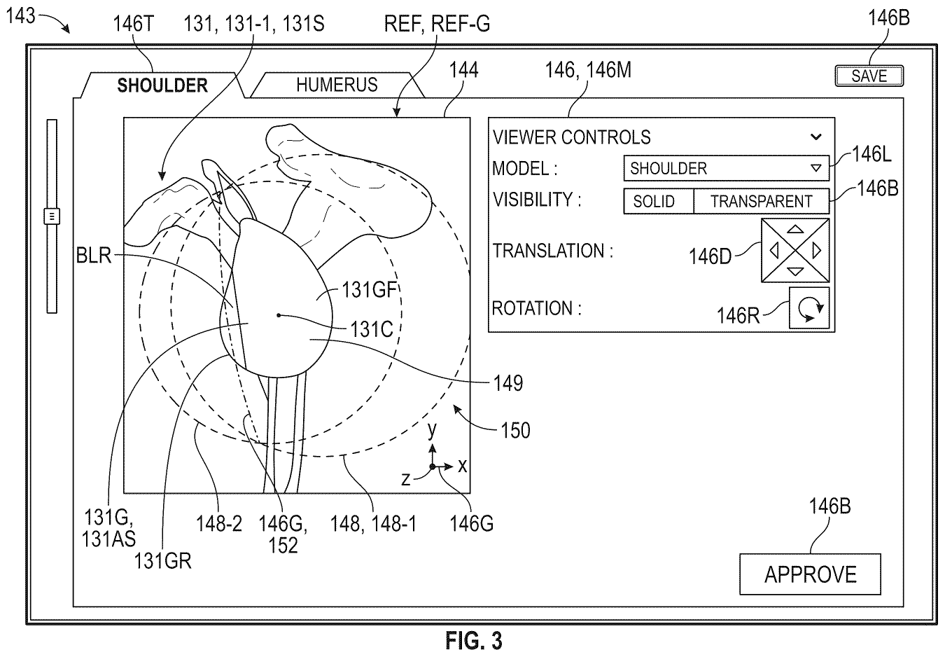

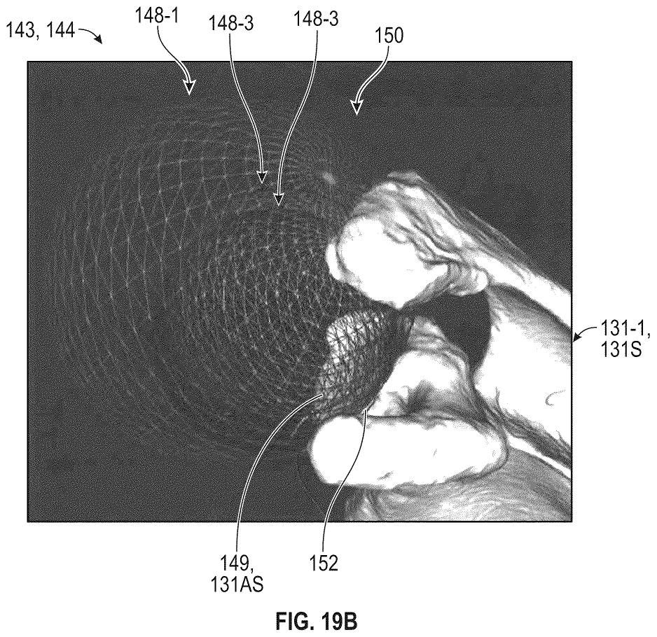

Referring to FIG. 3, with continuing reference to FIG. 2, the user interface 143 may include one or more display windows 144 and one or more objects 146. The objects 146 may include graphics such as menus, tabs and buttons accessible by user interaction, such as tabs 146T, buttons 146B, drop-down lists 146L, menus 146M, entry fields, directional indicators 146D, 146R and graphics 146G (e.g., intersecting ring 152). Geometric objects including selected anatomical model(s) 131, implant model(s) 132 (e.g., FIGS. 2 and 20A-B) and/or other information relating to the surgical plan 133 may be displayed in one or more of the display windows 144.

The display module 138 may be configured to display one or more selected anatomical models 131 and/or implant models 132 in the display windows 144. The display module 138 may be configured such that the selected anatomical model(s) 131 and/or implant model(s) 132 may be selectively displayed and hidden (e.g., toggled) in one or more of the display windows 144 in response to user interaction with the user interface 143, which may provide the surgeon with enhanced flexibility in reviewing aspects of the surgical plan 133.

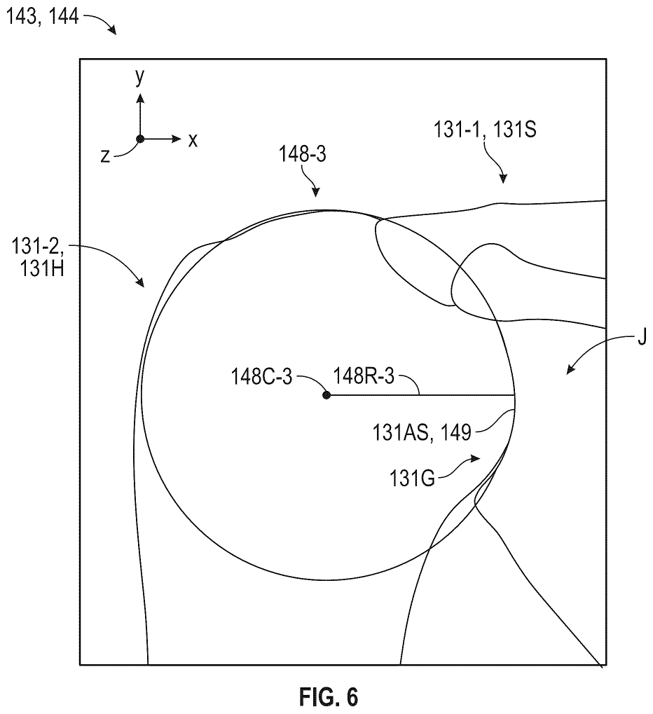

The data module 137 may be configured to access the anatomical model 131 from the database 129, which may occur automatically or in response to user interaction with the user interface 143. The data module 137 may be configured to store an instance of the selected anatomical model 131 in the memory 136. The anatomical model 131 may be associated with a joint. In the implementation of FIG. 6, the anatomical model(s) 131 may include a first virtual (e.g., 2D or 3D) anatomical model 131-1 and/or a second virtual (e.g., 2D or 3D) anatomical model 131-2. The first anatomical model 131-1 may be associated with a first bone of a patient. The second anatomical model 131-2 may be associated with a second (e.g., adjacent) bone of the patient. The data module 137 may be operable to access the anatomical model(s) 131-1, 131-2. In implementations, the second anatomical model 131-2 may be omitted.

In the implementation of FIG. 6, the first anatomical model 131-1 may include a 2D and/or 3D surface contour 149 associated with a socket of a joint J. The joint J may include any of the joints disclosed herein, such as a shoulder joint associated with a glenoid and humeral head of a patient. The first anatomical model 131-1 may be representative of a scapula associated with a shoulder joint of a patient. The first anatomical model 131-1 may be associated with a glenoid. The surface contour 149 may be established along an articular surface 131AS of the first anatomical model 131-1. The second bone may cooperate with the socket of the first bone to establish the joint J. The second anatomical model 131-2 may be representative of a humerus associated with the shoulder joint of the patient. The second anatomical model 132-2 may be associated with a humeral head.

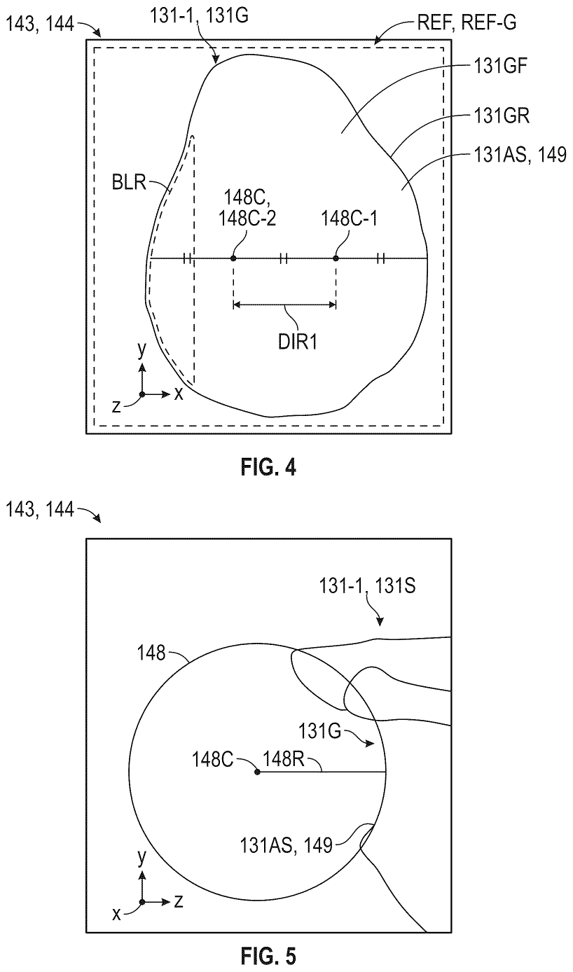

The display module 138 may be configured to display the selected anatomical model 131 in the display window(s) 144 of the user interface 143. The first anatomical model 131-1 may include a scapula model 131S associated with a scapula of the patient and/or a glenoid model 131G associated with a glenoid of the patient. The glenoid model 131G may include a glenoid face 131GF and/or glenoid rim 131GR (see also FIG. 4). The data model 137 and/or another portion of the planning environment 127 may be operable to access the glenoid model 131G. The second anatomical model 131-2 may be a humerus model 131H associated with a humerus of the patient. The humerus model 131H may be associated with a humeral head of the patient.

The display window(s) 144 may be configured to display a 2D and/or 3D representation of the selected anatomical model(s) 131. The anatomical model(s) 131 may be displayed with respect to X, Y and/or Z axes. The axes may be associated with respective anatomical planes of the patient. In implementations, the X axis may be associated with the A/P direction. The Y axis may be associated with the S/I direction. The Z axis may be associated with a medial/lateral (M/L) direction. The X, Y and/or Z axes may be associated with a coordinate graphic 146G. Although a particular number of display windows 144 are disclosed in the implementation of FIG. 3, it should be understood that the user interface 143 may be configured with any number of display windows 144 in accordance with the teachings disclosed herein, and aspects of the display windows 144 may be combined or separated.

The user interface 143 may be configured to display the anatomical model(s) 131 in various positions and/or orientations. The display window 144 may be configured to display a lateral view of the scapula, including the glenoid. In other implementations, the display window 144 may be configured to display a medial view, posterior view and/or anterior view of the scapula.

The surgeon or assistant may interact with the menu 146M, directly with the display window 144 and/or with another portion of the user interface 143 to move the selected anatomical model 131 in 2D space (e.g., up, down, left, right) and/or 3D space (e.g., rotation, tilt, zoom, etc.), which may occur in response to interaction with the directional indicators 146D, 146R and/or selection of the anatomical model 131 with an input device (e.g., cursor or touch screen). The surgeon or clinical user may interact with the coordinate graphic 146G to adjust (e.g., rotate or move) a viewing orientation of the anatomical model(s) 131.

The selected anatomical model 131 may include a 2D and/or 3D surface contour 149. The surface contour 149 may be associated with articular and/or non-articular surface(s) of the anatomy. The selected anatomical model 131 may include an articular surface 131AS. The surface contour 149 may be established by the articular surface 131AS. The glenoid model 131G may include the articular surface 131AS and/or surface contour 149. The articular surface 131AS may be associated with an articular surface of a bone, such as the glenoid, which may have an amount of bone loss or erosion. The bone loss may reside in a bone loss region BLR. The bone loss region BLR may reside along, or may otherwise be adjacent to, a perimeter of the articular surface 131AS, such as the glenoid rim 131GR.

The evaluation module 139 may be operable to determine (e.g., approximate) a location (e.g., direction) and/or severity (e.g., amount or magnitude) of bone loss associated with an articular surface of a bone corresponding to the selected anatomical model 131, such as bone loss associated with the bone loss region BLR. A profile of the glenoid model 131G may be representative of the erosion along the bone loss region BLR. Bone loss due to erosion may occur along a central portion and/or a periphery of the glenoid. The evaluation module 139 may be operable to determine one or more properties of the anatomical model 131, including the glenoid model 131G. The properties may include a location and/or amount of bone loss (e.g., erosion) along the bone loss region BLR.

The evaluation module 139 may be operable to generate one or more (e.g., 2D and/or 3D) spherical objects 148. The spherical objects 148 may have a substantially, or completely, spherical geometry. For the purposes of this disclosure the term “substantially” means ±10 percent of the stated value or relationship unless otherwise indicated. Each spherical object 148 may include one or more parameters, such as a respective center 148C (e.g., FIGS. 4-6) and/or radius 148R (e.g., FIG. 5). The display module 138 may be operable to display the spherical object(s) 148 in the display window(s) 144 and/or another portion of the user interface 143.

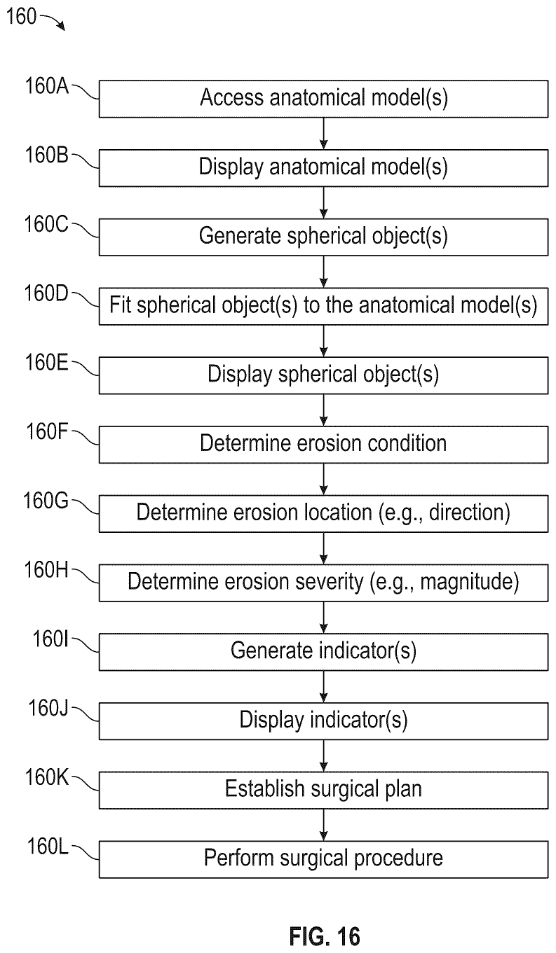

Still referring to FIG. 3, with continuing reference to FIG. 2, the spherical objects 148 may include a first spherical object 148-1 and/or a second spherical object 148-2. The display module 138 may be operable to display, in the display window 144 and/or another portion of the user interface 143, the spherical object(s) 148 relative to the surface contour 149 of the anatomical model 131. In implementations, the display module 138 may be operable to display, in the display window 144 and/or another portion of the graphical user interface 143, the first spherical object 148-1 and the second spherical object 148-2 relative to the surface contour 149 of the articular surface 131AS and/or glenoid model 131G. FIGS. 17A-17B disclose perspective (e.g., posterior and anterior) views of the first and second spherical objects 148-1, 148-2 (e.g., in wireframe) positioned relative to the surface contour 149 of the first anatomical model 131-1 in a display window 144 of the user interface 143.

The evaluation module 139 may be operable to set (e.g., initialize or instantiate) a position of the spherical object(s) 148 relative to the surface contour 149 of the anatomical model 131 utilizing various techniques. In the implementation of FIG. 4, the evaluation module 139 may be operable to instantiate the first and second spherical objects 148-1, 148-2 such that the respective centers 148C-1, 148C-2 of the first and second spherical objects 148-1, 148-2 may be distributed in a first direction DIR1 relative to the glenoid model 131G. The first direction DIR1 may be the A/P direction relative to the anatomy. The centers 148C-1, 148C-2 may be offset from each other in two or three dimensions based on the erosion condition (e.g., spherical objects 148-1, 148-2 of FIG. 3).

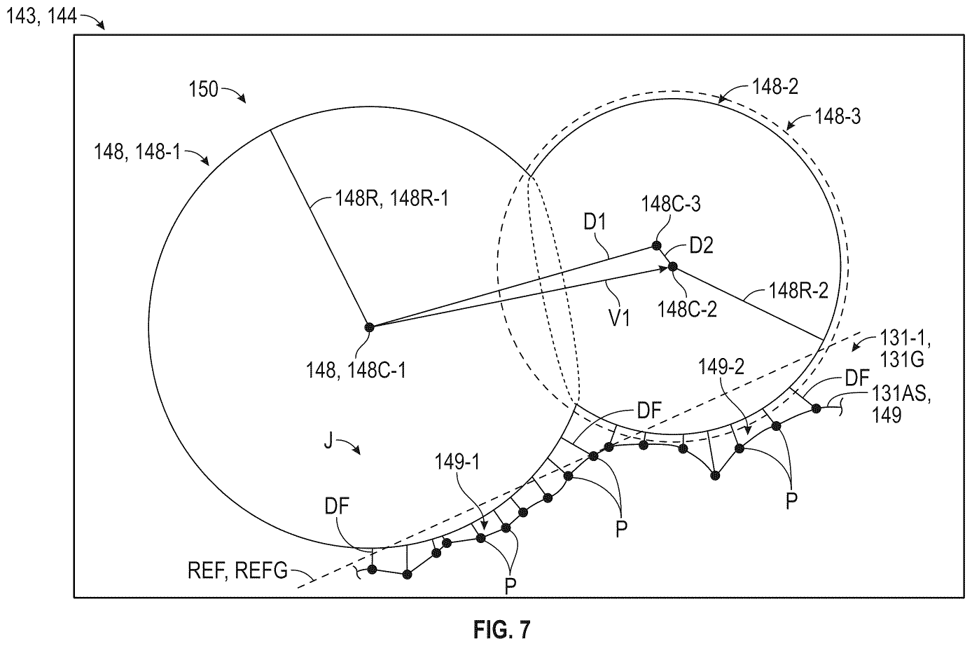

The evaluation module 139 may be operable to fit each spherical object 148 to a respective portion of the surface contour 149. In the implementation of FIG. 7, the surface contour 149 may include a first portion 149-1 and a second portion 149-2, which may extend from or may otherwise be adjacent to the first portion 149-1. The evaluation module 139 may be operable to fit the first spherical object 148-1 to the first portion 149-1 of the surface contour 149. The evaluation module 139 may be operable to fit the second spherical object 148-2 to the second portion 149-2 of the surface contour 149.

The evaluation module 139 may be operable to fit the spherical objects 148-1, 148-2 to the respective portions 149-1, 149-2 of the surface contour 149 such that a volume of the first spherical object 148-1 may (e.g., partially, but not completely) overlap with a volume of the second spherical object 148-2 (e.g., FIG. 3). The spherical objects 148-1, 148-2 may overlap to establish a 3D double intersecting (e.g., overlapping) spherical object 150. The evaluation module 139 may be operable to fit the spherical objects 148-1, 148-2 independently and/or may be operable to fit the spherical objects 148-1, 148-2 together as the overlapping spherical object 150. Fitting the spherical objects 148 may include adjusting one or more parameters of the spherical objects 148, such as the respective radius 148R, center 148C and/or position. The evaluation module 139 may be operable to generate and/or fit fewer or more than two spherical objects 148 to respective portions of the surface contour 149, such as only one spherical objects 148 or three or more spherical objects 148.

The surgeon or clinical user may interact with the menu 146M associated with the display window 144, directly with the display window 144 and/or another portion of the user interface 143 to adjust and/or otherwise set the parameter(s) including a position of the spherical object(s) 148 relative to each other and/or the anatomical model 131, including relative to the surface contour 149. The surgeon or clinical user may interact with a button 146B and/or another portion of the user interface 143 to approve a geometry and placement of the spherical object(s) 148.

The evaluation module 139 may be operable to determine a reference plane REF relative to the first anatomical model 131-1 (e.g., FIGS. 3-4). The reference plane REF may be associated with a periphery of a socket. In implementations, the periphery of the socket may be established by the glenoid rim 131GR. The reference plane REF may be a glenoid plane REF-G established relative to the glenoid model 131G. The glenoid plane REF-G may be associated with a profile of the glenoid.

The evaluation module 139 may be operable to fit the spherical object(s) 148 to the surface contour 149 of the anatomical model 131 utilizing various techniques, such as minimization (e.g., least squares). The evaluation module 139 may be configured to fit a periphery (e.g., boundary) of the spherical object 148 relative to a curvature of the surface contour 149, such as a curvature of the articular surface 131AS. The evaluation module 139 may be configured to execute one or more math libraries or functions to determine a fit between a curvature of the spherical object 148 relative to the curvature of the surface contour 149. The surgeon or clinical user may interact with the menu 146M, directly with the display window 144, and/or with another portion of the user interface 143 to adjust or set the shape, position and/or orientation of the spherical object(s) 148 and/or the overlapping spherical object 150 relative to the anatomical model 131.

Referring to FIG. 7, with continuing reference to FIGS. 2-6, the evaluation module 139 may be operable to fit the spherical objects 148-1, 148-2 and/or overlapping spherical object 150 to the anatomical model 131, which may occur in response to adjusting the respective sizes (e.g., radii 148R-1, 148R-2) and/or positions of the spherical objects 148-1, 148-2 to reduce distance(s) DF between adjacent surface points P along the surface contour 149 and the respective peripheries (e.g., surfaces) of the spherical objects 148-1, 148-2. The distances(s) DF may be minimum distances from the respective surface point P to the closest one of the spherical objects 148-1, 148-2. The spherical objects 148-1, 148-2 may be adjusted and the distances may be determined iteratively until a suitable fit is obtained.

The evaluation module 139 may be operable to determine an erosion condition associated with the surface contour 149 based on various aspects of the spherical object(s) 148 and/or overlapping spherical object 150. The first spherical object 148-1 may have a geometry that may approximate a native (e.g., paleo) curvature of the articular surface 131AS prior to the bone loss. The second spherical object 148-2 may have a geometry that may approximate a (e.g., neo) curvature of the articular surface 131AS associated with the bone loss.

The evaluation module 139 may be operable to determine an erosion condition associated with the surface contour 149 based on a relative size (e.g., volume) between the spherical objects 148-1, 148-2. The evaluation module 139 may be operable to determine the erosion condition based on the relative volume between the spherical objects 148-1, 148-2 being below a first preselected volume threshold (e.g., within 50% of each other).

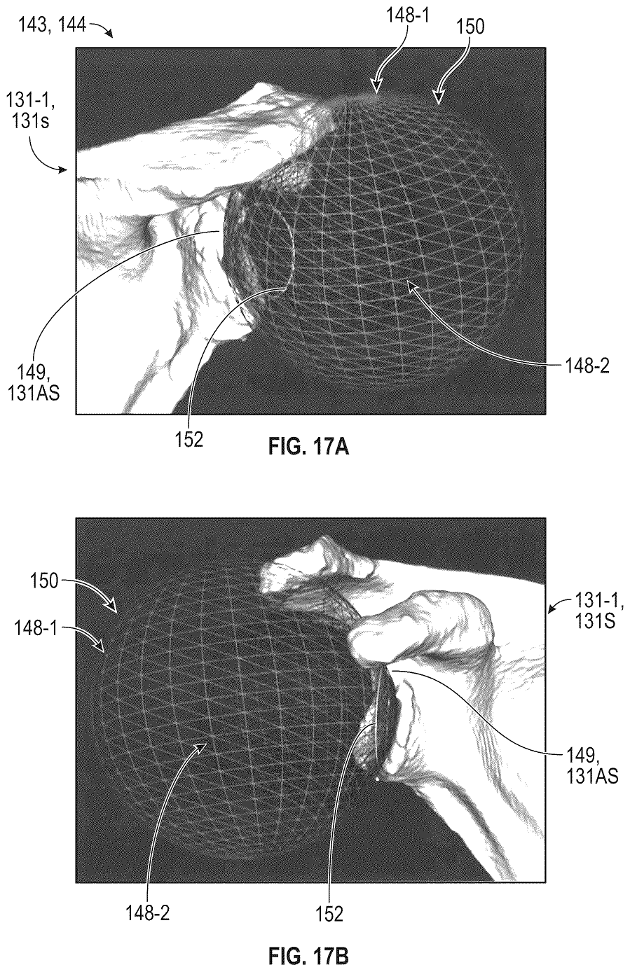

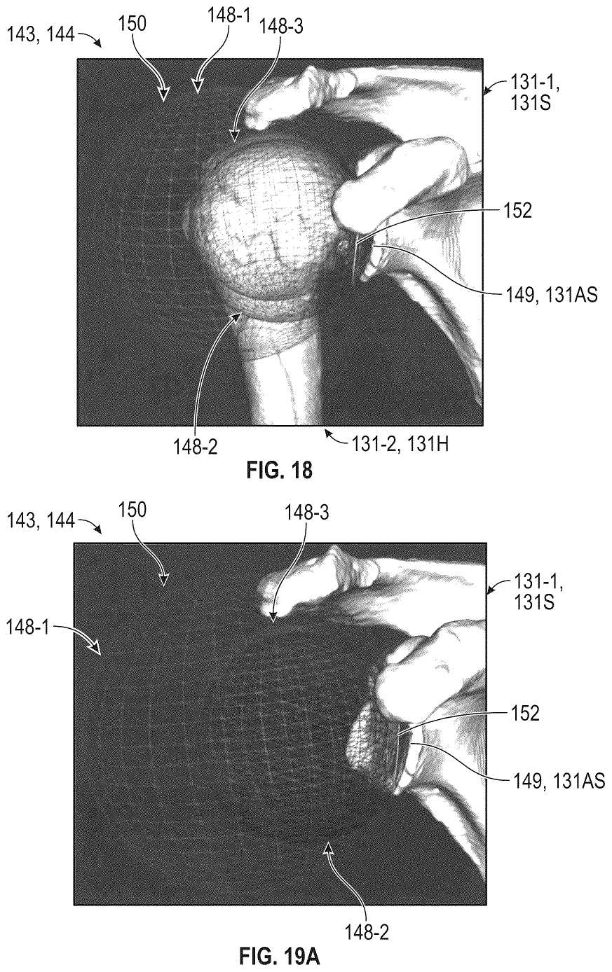

The evaluation module 139 may be operable to determine whether the first spherical object 148-1 or the second spherical object 148-2 may be associated with the erosion condition based on various characteristics of the humerus of the patient. In the implementation of FIG. 6, the spherical objects 148 may include a third spherical object 148-3. The evaluation module 139 may be operable to fit the third spherical object 148-3 to a volume of the second anatomical model 131-2, such as a volume associated with the humeral head. The evaluation module 139 may be operable to fit the third spherical object 148-3 to a portion of the humerus model 131H (see, e.g., FIG. 18), which may occur in response to adjusting the respective radius 148R-3 and/or center 148C-3 to reduce a distance between adjacent surface points of the humeral model 131H and the third spherical object 148-3. FIG. 18 discloses a perspective view of the first and second spherical objects 148-1, 148-2 (e.g., in wireframe) positioned relative to the first anatomical model 131-1, and the third spherical object 148-3 (e.g., in wireframe) positioned relative to the second anatomical model 131-2 in a display window 144 of the user interface 143. The third spherical object 148-3 may be fit to a portion of the first anatomical model 131-1, such as a portion of the humerus model 131H associated with a humeral head. FIGS. 19A-19B disclose perspective views of the first, second and third spherical objects 148-1, 148-2, 148-3 positioned relative to the first anatomical model 131-1 in a display window 144 of the user interface 143, with the second anatomical model 131-2 omitted.

Referring again to FIG. 7, the evaluation module 139 may be operable to determine an erosion condition associated with the surface contour 149 based on the sizes (e.g., volume) of the first and second spherical objects 148-1, 148-2 relative to the size (e.g., volume) of the third spherical object 148-3 (see, e.g., FIGS. 19A-19B). The evaluation module 139 may be operable to determine the erosion condition in response to a relative size (e.g., radius, diameter and/or volume) between one of the first and second spherical objects 148-1, 148-2 (e.g., object 148-2) and the third spherical object 148-3 meeting (e.g., being below) a second preselected size (e.g., volume) threshold. The curvature of the native glenoid cavity may be slightly larger than the curvature of the humeral head. In implementations, a contour of the erosion may be very close in size to a periphery of the third spherical object 148-3, which may be associated with the humerus.

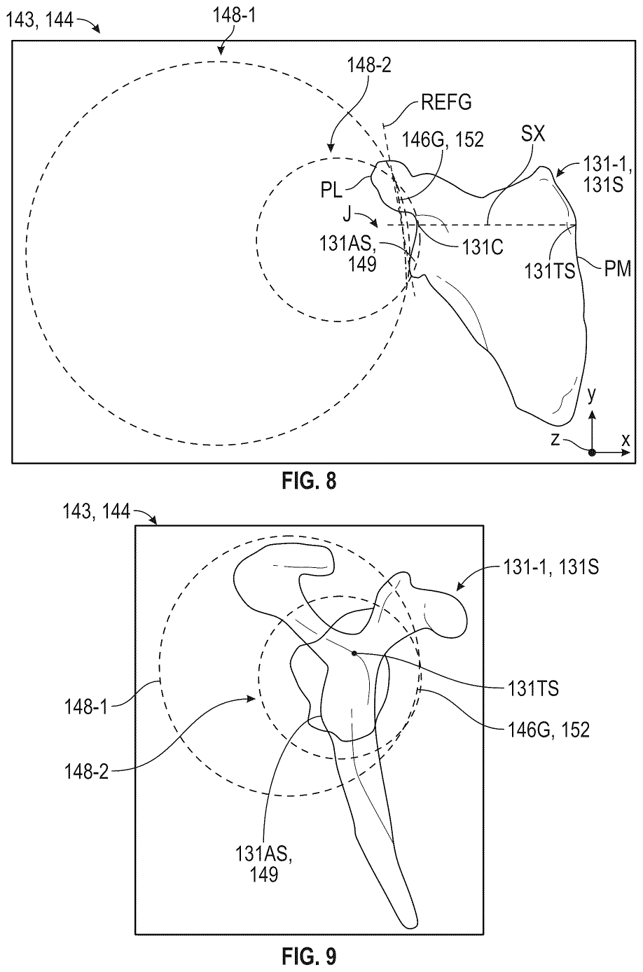

In the implementation of FIG. 8, the first spherical object 148-1 may be very large or very small (e.g., less than ⅓× or greater than 2×) relative to the scapula model 131S and/or second spherical object 148-2. In the implementation of FIG. 9, the spherical objects 148-1, 148-2 may be smaller in size relative to the implementation of FIG. 8. The spherical objects 148-1, 148-2 may be relatively closer in size to a size of the third spherical object 148-3 associated with a humeral head of the humeral model 131H (e.g., FIG. 7).

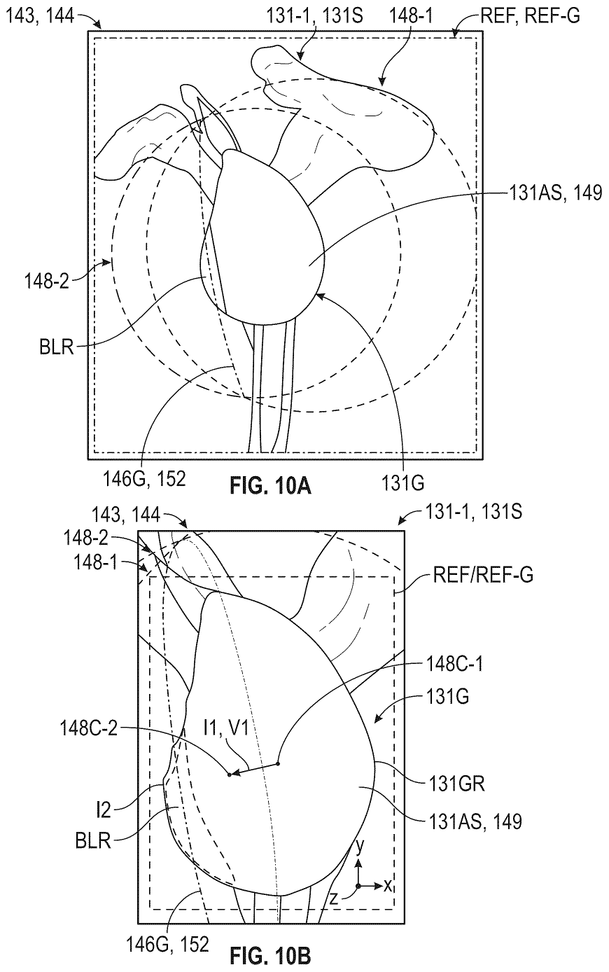

The evaluation module 139 may be operable to determine (e.g., approximate) a location of the erosion. Referring to FIGS. 10A-B and 11, with continuing reference to FIGS. 2-3, the evaluation module 139 may be operable to determine whether the erosion may be relatively central on the articular surface 131AS of the glenoid model 131G or may be due to misalignment of the humeral head associated with the humerus model 131H (e.g., FIGS. 6 and 18). The evaluation module 139 may be operable to determine whether the erosion condition may be associated with either central erosion or peripheral erosion based on the location of the spherical objects 148-1, 148-2 relative to the surface contour 149.

The evaluation module 139 may be operable to determine a location of erosion along the surface contour 149 associated with the erosion condition relative to the reference (e.g., glenoid) plane REF/REF-G. Glenoid version and/or inclination may be determined based on an orientation of the glenoid plane REF-G.

The evaluation module 139 may be operable to generate one or more intersecting rings 152 (see also FIGS. 3, 17A-17B, 18 and 19A-19B). The intersecting ring 152 may be generated along an intersection between the spherical objects 148-1, 148-2. The intersecting ring 152 may be generated along an intersection between a periphery of the first spherical object 148-1 and a periphery of the second spherical object 148-2. The display module 138 may be operable to display the intersecting ring 152 as a ring graphic 146G in the display window 144.

The evaluation module 139 may be operable to determine the location (e.g., direction) of the erosion based on an orientation of the intersecting ring 152 relative to the reference (e.g., glenoid) plane REF/REF-G. The evaluation module 139 may be operable to determine that the erosion may be adjacent to a central portion of the articular surface 131AS in response to determining that the intersecting ring 152 may be coplanar with, or may otherwise be substantially parallel to, the reference plane REF/REF-G (e.g., FIG. 11). The evaluation module 139 may be operable to determine that the erosion may be adjacent to a peripheral portion of the articular surface 131AS in response to determining that the intersecting ring 152 may be transverse to the reference plane REF/REF-G (e.g., FIGS. 10A-10B). In the implementation of FIG. 10A, the erosion may be peripheral to the center of the articular surface 131AS in an A/P direction and/or S/I direction. A slope of the reference plane REF/REF-G relative to the articular surface 131AS may indicate the direction of erosion.

The evaluation module 139 may be operable to determine whether the first spherical object 148-1 or the second spherical object 148-2 may be associated with peripheral erosion in response to determining the erosion condition (e.g., a best fit of the spherical objects 148 due to erosion). A portion of the surface contour 149 along the glenoid associated with the erosion may be referred to as the neo-glenoid surface. The neo-glenoid surface may be caused by erosion due to interaction of the articular surface with the (e.g., misaligned) humeral head. A geometry of the neo-glenoid surface may be associated with an imprint of the humeral head. The evaluation module 139 may be operable to select the spherical object 148-1/148-2 closest to a (e.g., humeral head) portion of the humeral model 131H as being associated with the neo-glenoid surface contour. In the implementation of FIG. 8, the anatomical model 131 may extend in the M/L direction between a first (e.g., most lateral) point PL and a second (e.g., most medial) point ML. The most medial point PM of the scapula model 131S may be established by the trigonum scapulae. The articular surface 131AS may be lateral of the most medial point PM with respect to the M/L direction. The evaluation module 139 may be operable to determine which of the spherical objects 148-1, 148-2 may be most inward (e.g., medial) relative to the articular surface 131AS and/or the associated anatomical model 131. The medial positions may be determined with respect to a peripheries and/or centers 148C-1, 148C-2 of the respective spherical objects 148-1, 148-2. The most inward spherical object 148-1/148-2 may be the spherical object 148 most inward from the joint J and/or surface contour 149 of the anatomical model 131-1 in a (e.g., M/L) direction opposite from an adjacent anatomical model 131 associated with the joint J (e.g., anatomical model 131-2 of FIG. 6). The evaluation module 139 may be operable to associate a most inward (e.g., medial) one of the first and second spherical objects 148-1, 148-2 with erosion along the surface contour 149. The evaluation module 139 may be operable to select the most inward (e.g., medial) spherical object 148-1/148-2 as being associated with the neo-glenoid surface contour. The most medial spherical object 148-1/148-2 may be closest to the most medial point PM. The evaluation module 139 may be operable to select the spherical object 148-1/148-2 as being associated with the neo-glenoid surface based on glenoid version and/or inclination. In implementations, retroversion values exceeding a preselected retroversion threshold may indicate posterior erosion, whereas anteversion values exceeding a preselected anteversion threshold may indicate anterior erosion. Retroversion/anteversion values exceeding the respective thresholds may indicate erosion, which may be determined independent of the humeral head location during imaging of the anatomy. The evaluation module 139 may be operable to determine whether either of the first and second spherical objects 148-1, 148-2 may be associated with peripheral erosion utilizing any of the techniques disclosed herein, either alone or in combination. In implementations, one of the techniques may be utilized to determine which of the first spherical object 148-1 or the second spherical object 148-2 may be associated with peripheral erosion, and one or more other techniques may be utilized to confirm (e.g., verify) the determination.

The evaluation module 139 may be operable to determine a location (e.g., direction) of peripheral erosion relative to the articular surface 131AS of the anatomical model 131. The evaluation module 139 may be operable to determine the location of the erosion relative to the A/P direction based on the determined glenoid version. The evaluation module 139 may be operable to determine posterior erosion of the glenoid in response to determining that the glenoid may be (e.g., highly) retroverted. The evaluation module 139 may be operable to determine a location of the erosion relative to the S/I direction based on the determined glenoid inclination. The evaluation module 139 may be operable to determine the severity of the erosion based on an amount of the determined version and/or inclination. A higher amount of version/inclination may be associated with more erosion than a relatively lesser amount of version/inclination.

The evaluation module 139 may be operable to determine the direction of the erosion relative to the center of the articular surface 131AS, including a center of the glenoid model 131G associated with the center of the glenoid face.

In the implementation of FIG. 7, the evaluation module 139 may be operable to determine a first distance D1 between the center 148C-1 of the first spherical object 148-1 and a center 148C-3 of the spherical object 148-3 associated with the humerus model 131H. The evaluation module 139 may be operable to determine a second distance D2 between the center 148C-2 of the second spherical object 148-2 and the center 148C-3 of the spherical object 148-3. The evaluation module 139 may be operable to associate one of the first and second spherical objects 148-1, 148-2 with erosion along the surface contour 149 corresponding to a lesser of the first and second distances D1, D2. In the implementation of FIG. 7, the second spherical object 148-2 may be associated with the erosion condition.

Referring to FIG. 10B, with continuing reference to FIGS. 2-3 and 7, the evaluation module 139 may be operable to determine a first vector V1. The first vector V1 may extend from the center 148C-1 of the first spherical object 148-1 to the center 148C-2 of the second spherical object 148-2 (e.g., in response to determining that the second spherical object 148-2 may be associated with the erosion condition), or vice versa. The evaluation module 139 may be operable to project the vector V1 onto the reference (e.g., glenoid) plane REF/REF-G. The evaluation module 139 may be operable to determine the erosion direction by projecting the first vector V1 onto the reference plane REF/REF-G. The evaluation module 139 may be operable to determine a direction of the erosion based on the projected vector V1 with respect to the reference plane REF/REF-G.

The evaluation module 139 may be operable to generate one or more indicators associated with the erosion condition. The display module 138 may be operable to display the indicator(s) in the display window(s) 144 and/or another portion of the user interface 143.

In the implementation of FIG. 10B, the display module 138 may be operable to display, in the display window 144 and/or another portion of the user interface 143, an indicator associated with a location (e.g., direction) and/or severity (e.g., magnitude) of erosion associated with the erosion condition. The display module 138 may be operable to display a first (e.g., directional) indicator I1 associated with the first vector V1 relative to the reference plane REF/REF-G in the display window 144. The first indicator I1 and/or first vector V1 may be associated with the determined direction of the erosion relative to the reference plane REF/REF-G. A length of the first indicator I1 and/or a length of the first vector V1 projected onto the reference plane REF/REF-G may be associated with an amount of (e.g., A/P and/or S/I) misalignment of the humeral head relative to the reference plane REF/REF-G. The indicators may include a second indicator I2 (shown in dashed lines), which may identify the bone loss region BLR. The first vector V1 may point in a direction of the bone loss region BLR.

The evaluation module 139 may be operable to determine a severity (e.g., magnitude) of the erosion associated with the erosion condition. Various techniques may be utilized to determine the severity. In an implementation, the evaluation module 139 may be operable to determine glenoid version and/or inclination based on an orientation of the glenoid plane REF-G. The evaluation module 139 may be operable to determine the severity based on the determined version and/or inclination.

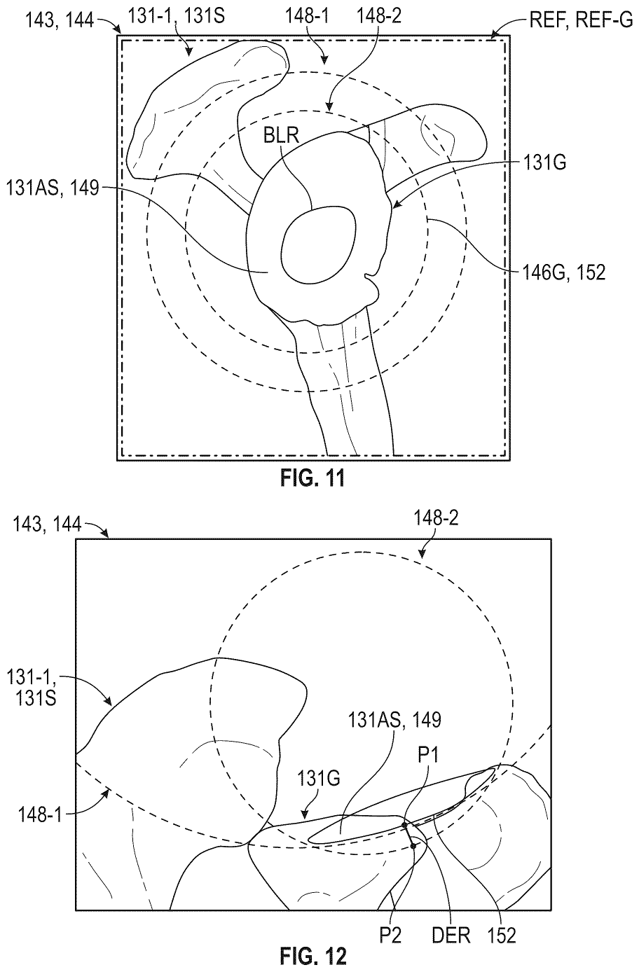

Referring to FIGS. 12-13, with continuing reference to FIGS. 2-3, the evaluation module 139 may be operable to determine the severity (e.g., magnitude) of the erosion based on a distance DER between a first point P1 on a periphery of the first (e.g., paleo-glenoid) spherical object 148-1 and a second point P2 on a periphery of the second (e.g., neo-glenoid) spherical object 148-2. The first and second points P1, P2 may be adjacent to the articular surface 131AS. The distance DER may be a maximum distance between the peripheries of the spherical objects 148-1, 148-2 adjacent to the articular surface 131AS and/or surface contour 149. A lesser distance DER may be associated with relatively lesser amount erosion. A greater distance DER may be associated with a relatively greater amount of erosion. If the points P1, P2 are relatively close, then the neo-glenoid spherical object 148-2 may be relatively close to the paleo-glenoid spherical object 148-1, which may be associated with relatively low erosion. However, if the points P1, P2 are relatively far, then the neo-glenoid spherical object 148-2 may be relatively far from the paleo-glenoid spherical object 148-1, which may be associated with a relatively high amount of erosion. The implementation of FIG. 12 may be associated with mild or medium erosion. The implementation of FIG. 13 may be associated with medium or severe erosion.

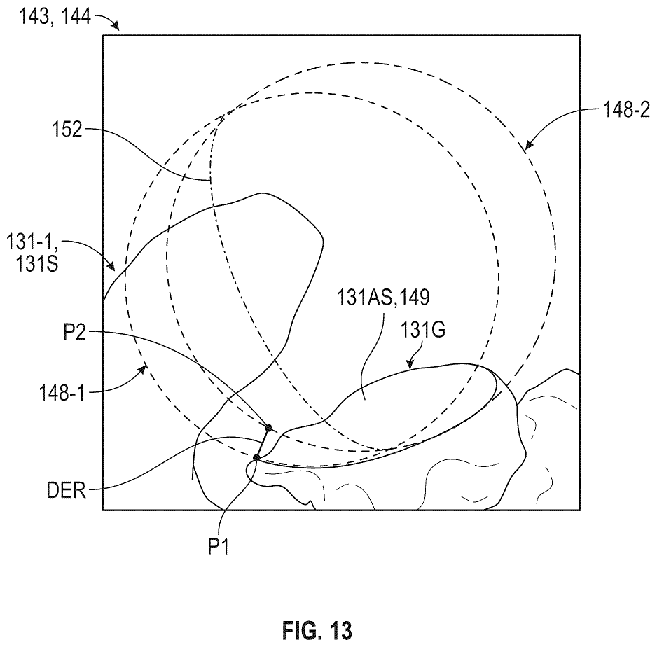

Referring to FIG. 14, with continuing reference to FIGS. 2-3 and 7, the first portion 149-1 of the surface contour 149 may be associated with a native glenoid surface. The second portion 149-2 of the surface contour 149 may be associated with an eroded glenoid surface. In implementations in which the determined erosion may be associated with the second spherical object 148-2, the evaluation module 139 may be operable to determine the severity (e.g., magnitude) of the erosion based on distances DP between respective surface points P along the second portion 149-2 of the surface contour 149 and a periphery of the first spherical object 148-1. In implementations in which the determined erosion may be associated with the first spherical object 148-1, the evaluation module 139 may be operable to determine the severity (e.g., magnitude) of the erosion based on distances DP between respective surface points P along the first portion 149-1 of the surface contour 149 and a periphery of the second spherical object 148-2 (e.g., FIG. 7). The magnitude may be associated with an average and/or total of the distances DP. The display module 138 may be operable to display the determined magnitude (e.g., score) and/or another indicator associated with the magnitude in a graphic 146G.

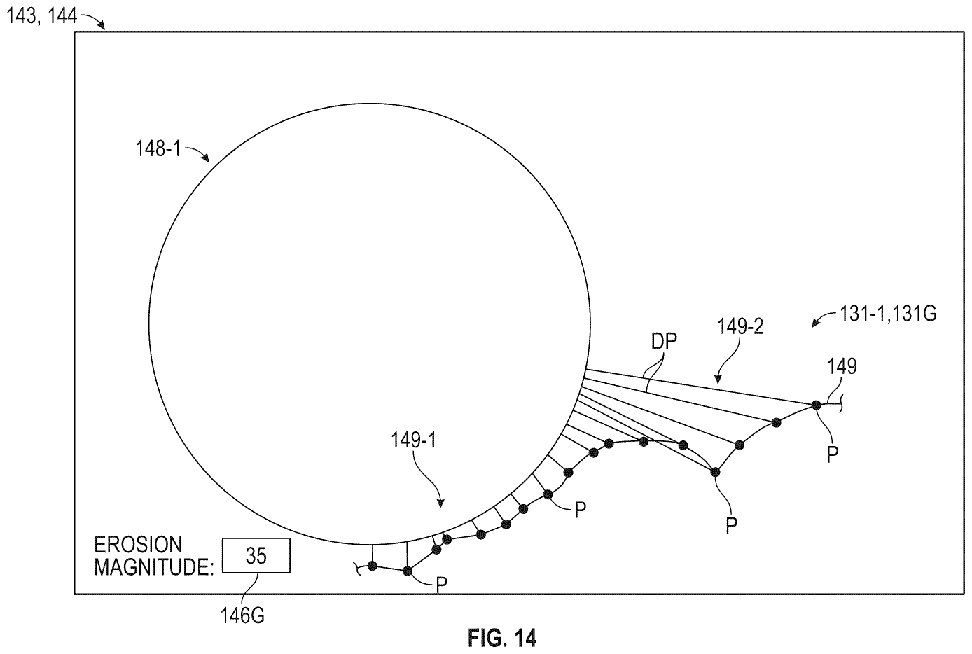

Referring to FIG. 15, with continuing reference to FIGS. 2-3 and 7, the evaluation module 139 may be operable to determine a depth of erosion along respective regions of the anatomical (e.g., glenoid) model 131-1/131G based on the first and/or second spherical objects 148-1, 148-2. The display module 138 may be operable to display, in the user interface 143, a heat map 154 associated with the depth of erosion. Various techniques may be utilized to represent the depth of erosion on the heat map 154. The depth of erosion may be displayed in the heat map 154 as a gradient associated with respective points along the surface contour 149. The gradient may be displayed in color or grayscale. Intensity and/or hue levels of the heat map 154 may be associated with the depth of erosion at point pairs between the spherical objects 148-1, 148-2 and the points P along the surface contour 149 of the articular surface 131AS (e.g., FIG. 7). The distances DF between the point pairs may establish the respective depths (e.g., FIG. 7). In implementations, the distance DER may between the points P1, P2 may representative one of the point pairs (e.g., FIGS. 12-13).

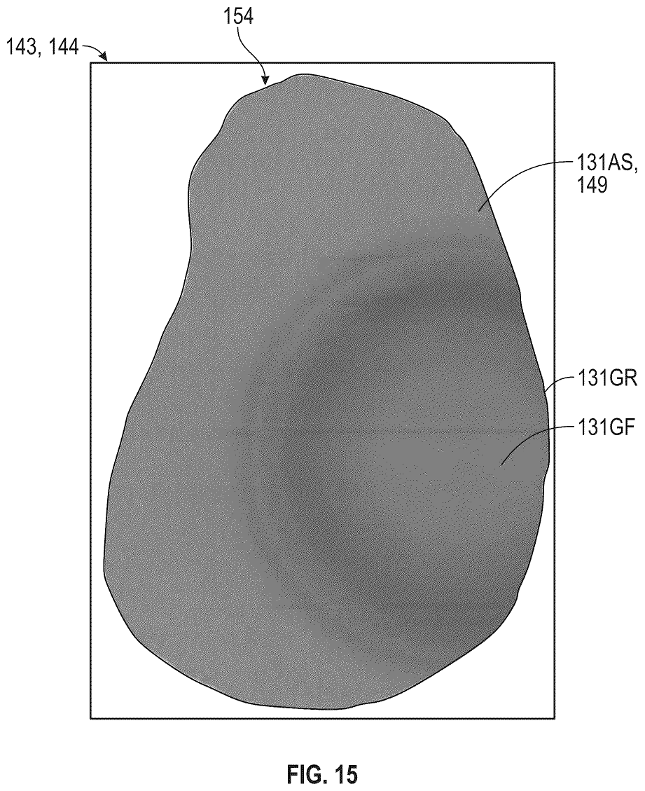

FIG. 16 discloses a method of planning an orthopaedic procedure in a flowchart 160 according to an implementation. The method 160 may be utilized to pre-operatively plan and perform an arthroplasty for restoring functionality to shoulders, ankles, knees, hips and other joints having bone loss or erosion along articular surfaces of the joint. The method 160 may be utilized to determine (e.g., estimate) a location (e.g., direction) and/or severity (e.g., magnitude) of bone loss associated with erosion along, or otherwise adjacent to, an articular surface of a bone, such as along a socket associated with a joint. The system 20/120 may be operable to perform any of the functionality of the method 160. Fewer or additional steps than are recited below could be performed within the scope of this disclosure, and the recited order of steps is not intended to limit this disclosure. Reference is made to the system 120.

Referring to FIGS. 2-3, with continuing reference to FIG. 16, one or more anatomical models 131 may be accessed at block 160A. The anatomical model 131 may be selected automatically and/or in response to user interaction with the graphical user interface 143. The anatomical models 131 may include a first anatomical model 131-1 (e.g., FIG. 3) and/or a second anatomical model 131-2 (e.g., FIG. 6). The anatomical model(s) 131-1, 131-2 may be associated with respective bones of a joint. The first anatomical model 131-1 may be associated with a glenoid of the shoulder joint. The second anatomical model 131-2 may be associated with a humerus of the shoulder joint, including a humeral head. The anatomical model(s) 131 may include a shoulder model 131S associated with a shoulder of the patient (e.g., FIG. 3) and/or a humerus model 131H associated with a humerus of the patient (e.g., FIG. 6).

Referring to FIG. 3, with continuing reference to FIGS. 2 and 16, at block 160B the selected anatomical model(s) 131 may be displayed in the user interface 143 of the planning environment 127. The anatomical model(s) 131 may be displayed in the user interface 143 utilizing any of the techniques disclosed herein.

At block 160C, one or more spherical objects 148 may be generated. The spherical objects 148 may include a first spherical object 148-1 and/or a second spherical object 148-2. The spherical objects 148-1, 148-2 may be associated with the scapula of the patient, including the glenoid. In implementations, the spherical objects 148 may include a third spherical object 148-3, which may be associated with the humerus of the patient (e.g., FIG. 6).

At block 160D, the spherical object(s) 148 may be fit relative to the anatomical model(s) 131. The spherical object(s) 148 may be fit relative to the anatomical model(s) 131 utilizing any of the techniques disclosed herein. In the implementation of FIGS. 7 and 17A-17B, block 160D may include fitting the first spherical object 148-1 to the first portion 149-1 of the (e.g., 2D or 3D) surface contour 149 of the first anatomical model 131-1. The first portion 149-1 may be associated with a socket of a joint J, such as the glenoid. Block 160D may include fitting the second spherical object 148-2 to the second portion 149-2 of the surface contour 149, which may be adjacent to the first portion 149-1 (e.g., FIGS. 6 and 18). Fitting the first and/or second spherical objects 148-1, 148-2 may include adjusting one or more parameters, including a radius 148R, center 148C and/or position of the respective spherical object 148, to minimize or otherwise reduce the distance(s) DF between surface points P along the surface contour 149 and the respective spherical object 148. The spherical objects 148-1, 148-2 may overlap to establish a (e.g., 3D) double intersecting (e.g., overlapping) spherical object 150.

At block 160E, the spherical object(s) 148 may be displayed in the user interface 143. The first and/or second spherical objects 148-1, 148-2 may be displayed relative to the surface contour 149 of the first anatomical model 131-1 (e.g., FIGS. 3, 7 and 17A-17B). The third spherical object 148-3 may be displayed relative to the second anatomical model 131-2 (e.g., FIGS. 6 and 18) and/or the first anatomical model 131-1 (e.g., FIGS. 7, 18 and 19A-19B). In implementations, the second anatomical model 131-2 may be omitted from the display of the third spherical object 148-3 (e.g., FIGS. 19A-19B). The surgeon or clinical user may interact with the user interface 143 to selectively display the second anatomical model 131-2 and/or third spherical object 148-3 relative to the first and/or second spherical objects 148-1, 148-2 and/or the first anatomical model 131-1 in one or more display windows 144 of the user interface 143.

At block 160F, an erosion condition associated with the surface contour 149 of the anatomical model 131 may be determined. The erosion condition may be determined utilizing any of the techniques disclosed herein. In implementations, the erosion condition associated with the surface contour 149 may be determined based on a relative size between the first, second and/or third spherical objects 148-1, 148-2, 148-3 (e.g., FIGS. 7-9, 17A-17B, 18 and 19A-19B).

The erosion condition may be determined based on one or more characteristics of an adjacent bone, such as the humerus. In the implementation of FIG. 6, block 160F may include fitting the third spherical object 148-3 to a three-dimensional surface contour of the second anatomical model 131-2. The second anatomical model 131-2 may be associated with a bone of the joint, such as the humerus. In the implementation of FIGS. 8-9, 18 and 19A-19B, determining the erosion condition may include determining whether a difference between a size (e.g., volume) of the third spherical object 148-3 (and/or the scapula model 131S) and a size (e.g., volume) of the first spherical object 148-1 may be within a first preselected limit. Determining the erosion condition may include determining whether a difference between the size (e.g., volume) of the third spherical object 148-3 and the size (e.g., volume) of the second spherical object 148-2 may be within a second preselected limit. In implementations, the erosion condition may be met in response to the difference(s) in the size(s) being below the first and/or second preselected limits. The erosion condition may not be met in response to the difference(s) in the size(s) meeting or exceeding the first and/or second preselected limits, which may be associated with fitting the spherical object 148 to outlier points (e.g., osteophytes or far rim points). In the implementation of FIG. 7, block 160F may include determining whether the first spherical object 148-1 or the second spherical object 148-2 may be associated with erosion along the surface contour 149 based on the distances D1, D2 between the respective centers 148C-1, 148C-2 of the first and second spherical objects 148-1, 148-2 and the center 148C-3 of the third spherical object 148-3. The spherical object 148-1/148-2 having the lesser distance D1/D2 may be associated with the erosion.

At block 160G, a location (e.g., direction) of the erosion associated with the erosion condition may be determined. The erosion location may be determined utilizing any of the techniques disclosed herein.

Referring to FIGS. 10A-B and 11, with continuing reference to FIGS. 2-3 and 16, block 160G may include determining a reference plane REF/REF-G relative to a rim of the articular surface 131AS of the first anatomical model 131-1. The articular surface 131AS may be associated with a periphery of a socket, such as the glenoid rim 131GR. Block 160G may include determining a location of erosion along the surface contour 149 associated with the erosion condition relative to the reference plane REF/REF-G. An intersecting ring 152 may be generated along an intersection between a periphery of the first spherical object 148-1 and a periphery of the second spherical object 148-2. The location of the erosion may be determined based on an orientation of the intersecting ring 152 relative to the reference plane REF/REF-G. In implementations, the erosion may be adjacent to a central portion of the articular surface 131AS in response to the intersecting ring 152 being coplanar with, or otherwise substantially parallel to, the reference plane REF/REF-G (e.g., FIG. 11). The erosion may be adjacent to a peripheral portion (e.g., rim) of the articular surface 131AS in response to the intersecting ring 152 being transverse, but not substantially parallel, to the reference plane REF/REF-G (e.g., FIGS. 10A-10B).

Referring to FIG. 10B, with continuing reference to FIGS. 2-3, 10A and 16, determining a direction of the erosion may include determining a first vector V1 from the center 148C-1 of the first spherical object 148-1 to the center 148C-2 of the second spherical object 148-2 (see also FIG. 7). The vector V1 may be projected onto the reference plane REF/REF-G. The direction of the erosion may be determined based on the projected vector V1. The direction may extend in the A/P direction and/or S/I direction. In implementations, the direction may extend in the M/L direction.

At block 160H, a severity (e.g., magnitude) of the erosion associated with the erosion condition may be determined. The erosion severity may be determined utilizing any of the techniques disclosed herein.

In the implementation of FIGS. 12-13, the severity (e.g., magnitude) of the erosion may be determined based on a distance DER between a first point P1 on the periphery of the first (e.g., paleo-glenoid) spherical object 148-1 and a second point P2 on a periphery of the second (e.g., neo-glenoid) spherical object 148-2. The first and second points P1, P2 may be adjacent to the articular surface 131AS. The distance DER may be a maximum distance between the peripheries of the spherical objects 148-1, 148-2 adjacent to the articular surface 131AS and/or surface contour 149. A lesser distance DER may be associated with relatively lesser amount erosion. A greater distance DER may be associated with a relatively greater amount of erosion.

In the implementation of FIG. 14, the severity (e.g., magnitude) of the erosion may be determined based on the distances DP between the respective surface points P along the second portion 149-2 of the surface contour 149 and the periphery of the first spherical object 148-1. In other implementations, the magnitude of the erosion may be determined based on distances between respective surface points P along the first portion 149-1 of the surface contour 149 and a periphery of the second spherical object 148-2 (e.g., if the determined erosion extends along the first portion 149-1 of the surface contour 149).

At block 160I, one or more indicators associated with the erosion may be generated. The indicators may include any of the indicators disclosed herein and may be generated utilizing any of the techniques disclosed herein. The indicator(s) may be associated with a location (e.g., direction) and/or severity (e.g., magnitude) of erosion associated with the erosion condition (e.g., indicators I1, I2 of FIG. 10B).

At block 160J, the indicator(s) may be displayed in the user interface 143 (e.g., FIG. 10B). The indicator(s) may be displayed utilizing any of the techniques disclosed herein. In the implementation of FIG. 15, a heat map 154 may be displayed in the user interface 143. The heat map 154 may be associated with a depth of erosion along respective regions (e.g., points) of the surface contour 149 based on the determined erosion condition.

At block 160K, a surgical plan 133 for treating the patient may be established based on the determined erosion condition. Block 160K may include selecting one or more implants and/or bone grafts for treating the joint based on the determined erosion condition. In the implementation of FIGS. 20A-20B, a virtual 2D and/or 3D implant model 132 associated with the selected implant may be positioned relative to the surface contour 149 of the anatomical model 131-1 based on the determined erosion condition. In implementations, a portion of tissue may be removed from the surface contour 149 prior to positioning the implant model 132. The implant model 132 may be patient-specific or may be generic.

At block 160L, the surgeon may perform a surgical procedure based on the surgical plan 133. Block 160L may include any of the surgical procedures disclosed herein. The surgeon may position one or more implants and/or bone grafts relative to the anatomy of the patient, which may treat the erosion condition.

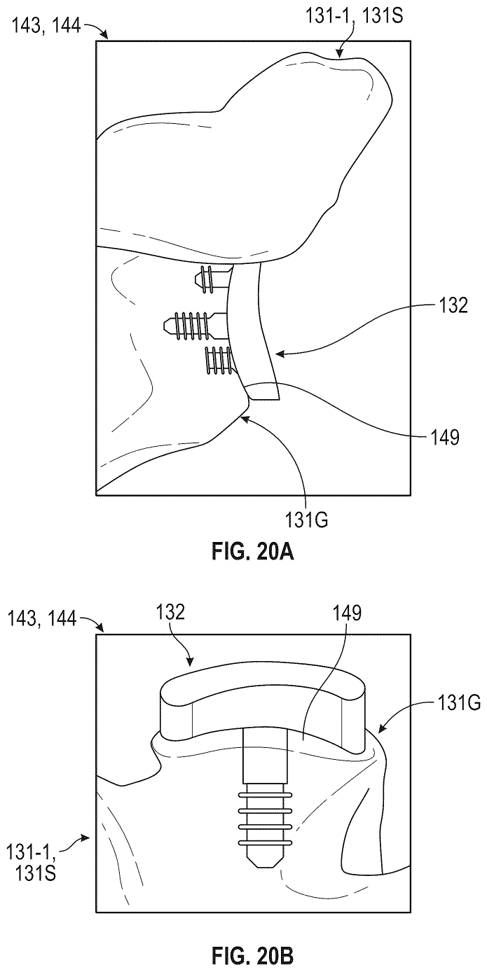

Referring to FIG. 21, with continuing reference to FIGS. 2-3, 6-7, 18, 19A-19B and 21, the evaluation module 139 may be operable to determine one or more parameters (e.g., measurements), which may be associated with wear (e.g., erosion). The evaluation module 139 may be operable to determine the parameter(s) based on any of the features and/or other information disclosed herein, including the spherical object(s) 148, determined erosion condition which may include the direction and/or magnitude of the erosion, and/or information used to determine the erosion condition. The evaluation module 139 may be operable to determine (e.g., assign or calculate) values for the parameter(s).

The display module 138 may be operable to display the parameter(s) in one or more display windows 144 and/or another portion of the user interface 143. The display windows 144 may include a first display window 144-1, a second display window 144-2, a third display window 144-3 and/or a fourth display window 144-4. In implementations, the first display window 144-1 may be associated with glenoid orientation. The parameters may be associated with an orientation of the glenoid, which may include a version and/or an inclination of the glenoid. The version and/or inclination may be determined based on an orientation of the glenoid plane REFG relative to a scapula axis SX (e.g., FIGS. 7-8). The scapula axis SX may extend between a center 131C of the articular surface 131AS and a center of a trigonum scapulae 131TS of the scapula model 131S (e.g., FIGS. 3 and 8-9).

The second display window 144-2 may be associated with joint metric(s). The joint metrics may be established with respect to the scapula axis SX. The metrics may include a subluxation index, which may include values associated with the scapula axis SX, a mediatrice and/or critical shoulder angle. The mediatrice may quantify posterior humeral head subluxation using a line perpendicular to the glenoid face.

The third display window 144-3 may be associated with glenoid wear (e.g., erosion). The glenoid erosion may be associated with a relative concavity, which may characterize a profile (e.g., flattening) of the glenoid face. Glenoid concavity flattening may cause the cup-shaped glenoid to become relatively flat, which may occur due to repetitive dislocations and/or bone loss. Glenoid flattening may reduce the concavity compression that may stabilize the shoulder joint.

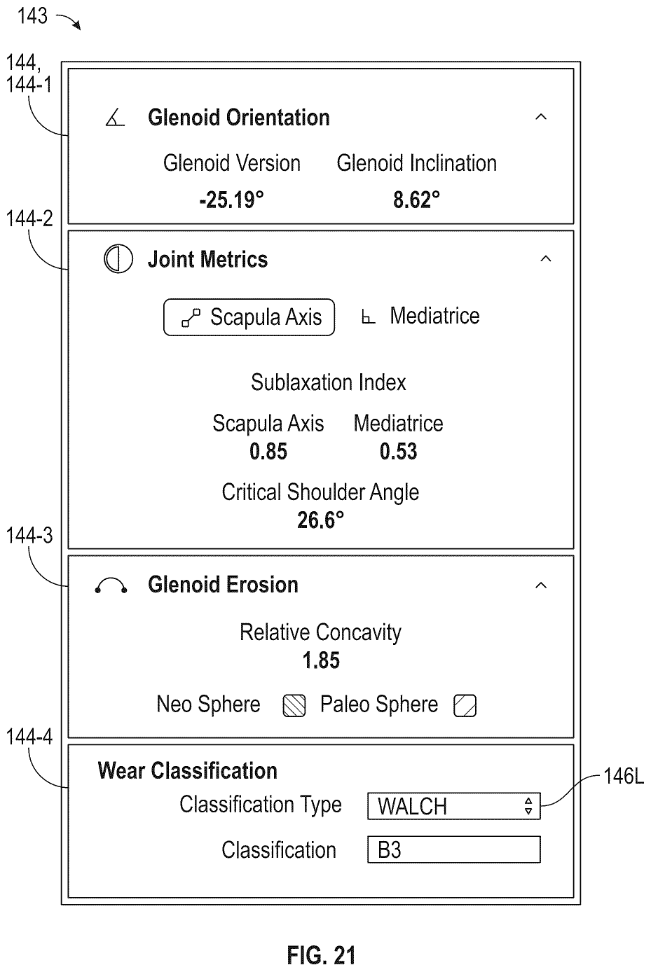

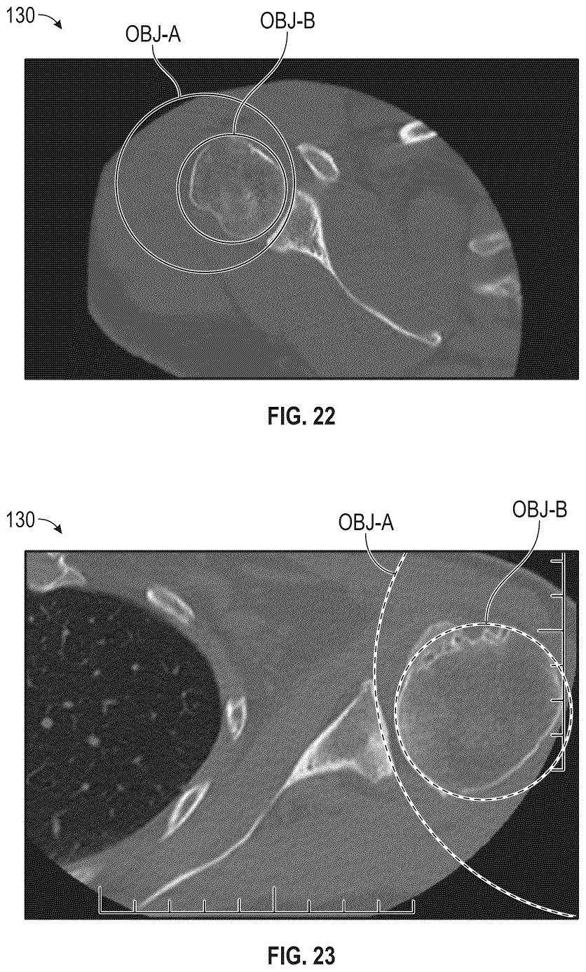

The evaluation module 139 may be operable to determine a relative concavity associated with a profile of the surface contour of the anatomical model 131, which may include the glenoid model 131G. Various techniques may be utilized to determine relative concavity. Relative concavity may be defined as a radius 148R-3 of the third (e.g., humeral head) spherical object 148-3 associated with the humerus model 131H divided by a radius 148R-1, 148-2 of one of the first and second spherical objects 148-1, 148-2 associated with the glenoid model 131G, such as the first (e.g., paleo) spherical object 148-1 (e.g., FIGS. 6-8). In the implementation of FIGS. 7-8, the paleo spherical object 148 may be the first spherical object 148-1. In the images 130 of FIGS. 22-23, a first object OBJ-A may be associated with the humeral head spherical object 148-3. A second object OBJ-B may be associated with the paleo spherical object 148-1. In the image 130 of FIG. 22, the radii of the objects OBJ-A, OBJ-B may be relatively close in size, which may be associated with a glenoid lacking wear or having reduced wear. In the image 130 of FIG. 23, a difference between the radii of the objects OBJ-A, OBJ-B may be relatively greater than in the image 130 of FIG. 22, which may be indicative of flattening of the glenoid.

The fourth display window 144-4 may be associated with a (e.g., wear or erosion) classification. The evaluation module 139 may be operable to determine the classification of the anatomical model 131 based on the parameter(s). The classification may include any of the classifications techniques disclosed herein, including a Walch classification associated with glenoid erosion. The evaluation module 139 may be operable to determine (e.g., assign) the classification based on parameter(s), which may include the determined (e.g., assigned or calculated) glenoid orientation, subluxation and/or relative concavity. The display module 138 may be operable to display the determined classification in the display window 144-4 and/or another portion of the user interface 143.

The surgeon or clinical user may interact with a drop-down list 146L and/or another portion of the user interface 143 to select a classification technique, which may be associated with wear (e.g., erosion). The classification technique may be selected from a set of (e.g., predefined) classification techniques, including any of the classification techniques disclosed herein such as the Walch and/or Favard classification systems. The evaluation module 139 may be operable to determine (e.g., assign) the selected classification based on the parameter(s) associated with the anatomical model 131. The display module 138 may be operable to display the determined classification in the display window 144-4 and/or another portion of the user interface 143.

The surgeon or clinical user may interact directly with the display window(s) 144 and/or another portion of the user interface 143 to adjust a placement of the spherical object(s) 148 and/or a relative fit between the spherical object(s) 148 and the articular surface 131AS (e.g., FIGS. 6-7, 17A-19C). The evaluation module 139 may be operable to determine the parameter(s) and/or classification based on the adjusted placement of the spherical object(s) 148. The display module 138 may be operable to update a display of the parameter(s) and/or the classification in response to the adjustment(s).

The evaluation module 139 may be operable to generate a surgical plan 131 based on the determined parameter(s) and/or classification. In implementations, method 160 may include determining the parameter(s) and/or classification (FIG. 16). Establishing the surgical plan 131 at block 160K may be based on the determined parameter(s) and/or classification.

The systems and methods disclosed herein may be utilized to classify erosion in two and/or three dimensions relative to an articular surface of the anatomy, such as the glenoid. A set of 3D spherical objects may be fit to the 3D surface contour of the articular surface to determine the erosion. The spherical objects may overlap to establish an overlapping spherical object. The location (e.g., direction) and/or severity (e.g., magnitude) of the erosion may be determined based on the spherical objects. The determined erosion may be utilized to establish a surgical plan for treating the patient, including selecting and/or precisely positioning an implant or bone graft, which may improve mobility and healing of the patient.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

1. A system for planning an orthopaedic procedure comprising:
a computing device including one or more processors coupled to memory, wherein the one or more processors are operable to collectively execute a planning environment, and the planning environment is operable to:
access a virtual three-dimensional glenoid model associated with a glenoid of a patient, the glenoid model including a three-dimensional surface contour;
fit a first three-dimensional spherical object to a first portion of the surface contour;
fit a second three-dimensional spherical object to a second portion of the surface contour such that a volume of the first spherical object overlaps with a volume of the second spherical object; and
determine an erosion condition associated with the surface contour based on a relative size between the first and second spherical objects.

2. The system as recited in claim 1, wherein the planning environment is operable to:
instantiate the first and second spherical objects such that respective centers of the first and second spherical objects are distributed in a first direction relative to the glenoid model.

3. (canceled)

4. The system as recited in claim 1, wherein the planning environment is operable to:
fit the first and second spherical objects in response to adjusting a respective radius or position to reduce a distance between adjacent surface points along the surface contour and the first and second spherical objects.

5. (canceled)

6. The system as recited in claim 1, wherein the planning environment is operable to:
access a virtual three-dimensional humerus model associated with a humeral head of the patient;
determine a first distance between a center of the first spherical object and a center of the humerus model;
determine a second distance between a center of the second spherical object and the center of the humerus model; and
associate one of the first and second spherical objects with erosion along the surface contour corresponding to a lesser of the first and second distances.

7. The system as recited in claim 1, wherein the planning environment is operable to:
determine the erosion condition based on a relative volume between the first and second spherical objects being below a first preselected volume threshold.

8. The system as recited in claim 7, wherein the planning environment is operable to:
access a virtual three-dimensional humerus model associated with a humeral head of the patient;
fit a third three-dimensional spherical object to the humerus model; and
determine the erosion condition in response to a relative volume between the second and third spherical objects meeting a second preselected volume threshold.

9. The system as recited in claim 8, wherein the planning environment is operable to:
determine a relative concavity associated with a profile of the surface contour of the glenoid model, wherein the relative concavity is defined as a radius of the third three-dimensional spherical object divided by a radius of one of the first and second spherical objects;
determine a wear classification based on the relative concavity; and
display the wear classification in a graphical user interface.

10. (canceled)

11. The system as recited in claim 1, wherein the planning environment is operable to:
determine a glenoid plane relative to the glenoid model, the glenoid plane associated with a profile of the glenoid; and
determine a location of erosion along the surface contour associated with the erosion condition relative to the glenoid plane.

12. The system as recited in claim 11, wherein the planning environment is operable to:
generate an intersecting ring along an intersection between a periphery of the first spherical object and a periphery of the second spherical object; and
determine the location of the erosion based on an orientation of the intersecting ring relative to the glenoid plane.

13. The system as recited in claim 11, wherein the planning environment is operable to:
determine a vector from a center of the first spherical object to a center of the second spherical object;
project the vector onto the glenoid plane; and
determine a direction of the erosion based on the projected vector.

14. The system as recited in claim 1, wherein the first portion of the surface contour is associated with a native glenoid surface, the second portion of the surface contour is associated with an eroded glenoid surface, and the planning environment is operable to:
determine a magnitude of the erosion based on distances between respective surface points along the second portion of the surface contour and a periphery of the first spherical object.

15-18. (canceled)

19. A system for planning an orthopaedic procedure comprising:
a computing device including one or more processors coupled to memory, wherein the one or more processors are operable to collectively execute a planning environment, and the planning environment is operable to:
access a first virtual three-dimensional anatomical model associated with a first bone of a patient, the first virtual anatomical model including a three-dimensional surface contour associated with a socket of a joint;
access a second virtual three-dimensional anatomical model associated with a second bone that cooperates with the socket of the first bone to establish the joint;
fit a first three-dimensional spherical object to a first portion of the surface contour;
fit a second three-dimensional spherical object to a second portion of the surface contour such that a volume of the first spherical object overlaps with a volume of the second spherical object;
fit a third spherical object to a volume of the second anatomical model; and
determine an erosion condition associated with the surface contour based on a volume of the first and second spherical objects relative to a volume of the third spherical object.

20. The system as recited in claim 19, wherein the planning environment is operable to:
generate an indicator associated with the erosion condition; and
display the indicator in a graphical user interface.

21-25. (canceled)

26. A method of planning an orthopaedic procedure comprising:
fitting a first spherical object to a first portion of a three-dimensional surface contour of a first virtual three-dimensional anatomical model, wherein the first portion is associated with a socket of a joint;
fitting a second spherical object to a second portion of the surface contour adjacent to the first portion;
determining an erosion condition associated with the surface contour based on a relative size between the first and second spherical objects;
displaying, in a graphical user interface, the first and second spherical objects relative to the surface contour of the first anatomical model; and
displaying, in the graphical user interface, an indicator associated with the erosion condition.

27. The method as recited in claim 26, wherein the steps of fitting the first and second spherical objects comprise:
adjusting a radius or position of the first or second spherical objects to reduce a distance between surface points along the surface contour and the first or second spherical objects.

28. The method as recited in claim 26, further comprising:
determining a reference plane relative to a rim of the first anatomical model associated with a periphery of the socket; and
determining a location of erosion along the surface contour associated with the erosion condition relative to the reference plane.

29. The method as recited in claim 28, further comprising:
generating an intersecting ring along an intersection between a periphery of the first spherical object and a periphery of the second spherical object; and
determining the location of the erosion based on an orientation of the intersecting ring relative to the reference plane.

30. The method as recited in claim 29, further comprising:
determining a vector from a center of the first spherical object to a center of the second spherical object;
projecting the vector onto the reference plane;
determining a direction of the erosion based on the projected vector; and
determining a magnitude of the erosion based on distances between respective surface points along the second portion of the surface contour and the periphery of the first spherical object;
wherein the indicator is associated with the direction or the magnitude of erosion associated with the erosion condition.

31-33. (canceled)

34. The method as recited in claim 26, further comprising:
selecting an implant for treating the joint based on the determined erosion condition; and
positioning a three-dimensional virtual implant model associated with the selected implant relative to the surface contour based on the determined erosion condition.

35-36. (canceled)

37. The method as recited in claim 26, further comprising:
fitting a third spherical object to a three-dimensional surface contour of a second virtual three-dimensional anatomical model, wherein the second anatomical model is associated with a bone of the joint;
wherein the step of determining the erosion condition comprises:
determining whether a difference between a volume of the third spherical object and a volume of the first spherical object is within a first preselected limit; or
determining whether a difference between the volume of the third spherical object and a volume of the second spherical object is within a second preselected limit.

38-39. (canceled)
